(19) United States
(12) Patent Application Publication
(10) Pub. No.: US 2024/0315991 A1
(43) Pub. Date: Sep. 26, 2024

(54) USES OF HNF4 ALPHA AGONISTS

(71) Applicant: Sanford Burnham Prebys Medical Discovery Institute, La Jolla, CA (US)

(72) Inventor: Fred LEVINE, Del Mar, CA (US)

(21) Appl. No.: 18/415,255

(22) Filed: Jan. 17, 2024

Related U.S. Application Data
(60) Provisional application No. 63/480,927, filed on Jan. 20, 2023.

(51) Int. Cl. A61K 31/165; A61P 1/16; A61P 3/04; C12Q 1/48; C12Q 1/6851

(52) U.S. Cl. CPC A61K 31/165; A61P 1/16; A61P 3/04; C12Q 1/48; C12Q 1/6851

(57) ABSTRACT

Provided herein are methods for using HNF4α agonists and pharmaceutical compositions thereof for reducing body weight, maintaining body weight, reducing diet induced weight gain, reducing mitochondrial stress, treating diseases or disorders such as inflammation, diabetes, nonalcoholic fatty liver disease (NAFLD) or nonalcoholic steatohepatitis (NASH) in a subject.

### CROSS REFERENCE

Any and all priority claims identified in the Application Data sheet, or any correction thereto, are hereby incorporated by reference under 37 CFR 1.57. For example, this Application claims the benefit of U.S. Provisional App. No. 63/480,927 filed on Jan. 20, 2023, which is incorporated by reference in its entirety herein.

### BACKGROUND

HNF4α is a nuclear receptor transcription factor that controls the expression of downstream genes that are important in multiple aspects of cellular metabolism. The classical view of HNF4α has been that its ligand binding pocket (LBP) is constitutively occupied by a fatty acid that plays a structural rather than regulatory role. However, it has been shown recently that the fatty acids in the HNF4α LBP are exchangeable in the context of full length HNF4α, particularly inside the cell.

Non-alcoholic fatty liver disease (NAFLD) affects about 20 to 25% of population. The annual economic burden is over US$100 billion in the US in 2016. Weight loss is the most effective treatment for NAFLD, however, no medicines specifically for NAFLD or nonalcoholic steatohepatitis (NASH) had received approval.

### SUMMARY

The present disclosure provides a method for reducing body weight of a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for reducing body weight and treating nonalcoholic fatty liver disease (NAFLD) in a subject in need thereof, comprising administering N-trans caffeoyltyramine, or a pharmaceutically acceptable salt thereof, to the subject in an amount of 30 mg per kg per day to about 400 mg per kg per day for a period of time. The present disclosure also provides a method for reducing body weight and treating nonalcoholic steatohepatitis (NASH) in a subject in need thereof, comprising administering N-trans caffeoyltyramine, or a pharmaceutically acceptable salt thereof, to the subject in an amount of 30 mg per kg per day to about 400 mg per kg per day for a period of time. The present disclosure also provides a method for reducing body weight and treating diabetes in a subject in need thereof, comprising administering N-trans caffeoyltyramine, or a pharmaceutically acceptable salt thereof, to the subject in an amount of 30 mg per kg per day to about 400 mg per kg per day for a period of time. The present disclosure also provides a method for reducing body weight and increasing fatty acid oxidation (FAO) activity in a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for treating obesity in a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for reducing body weight and reducing mitochondrial stress in a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for reducing body weight and treating inflammation in a subject in need thereof, comprising administering N-trans caffeoyltyramine, or a pharmaceutically acceptable salt thereof, to the subject in an amount of 30 mg per kg per day to about 400 mg per kg per day for a period of time. In some embodiments, wherein the level of IL-6, TNFα, or nitric oxide in the subject is decreased. The present disclosure also provides a method for maintaining weight of a subject or reducing diet induced weight gain of a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for maintaining weight of a subject or reducing diet induced weight gain of a subject and treating nonalcoholic fatty liver disease (NAFLD) in a subject in need thereof, comprising administering N-trans caffeoyltyramine, or a pharmaceutically acceptable salt thereof, to the subject in an amount of 30 mg per kg per day to about 400 mg per kg per day for a period of time. The present disclosure also provides a method for maintaining weight of a subject or reducing diet induced weight gain of a subject and treating nonalcoholic steatohepatitis (NASH) in a subject in need thereof, comprising administering N-trans caffeoyltyramine, or a pharmaceutically acceptable salt thereof, to the subject in an amount of 30 mg per kg per day to about 400 mg per kg per day for a period of time. The present disclosure also provides a method for maintaining weight of a subject or reducing diet induced weight gain of a subject and treating diabetes in a subject in need thereof, comprising administering N-trans caffeoyltyramine, or a pharmaceutically acceptable salt thereof, to the subject in an amount of 30 mg per kg per day to about 400 mg per kg per day for a period of time. The present disclosure also provides a method for maintaining weight of a subject or reducing diet induced weight gain of a subject and increasing fatty acid oxidation (FAO) activity in a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for maintaining weight of a subject or reducing diet induced weight gain of a subject and reducing mitochondrial stress in a subject, comprising administering N-trans caffeoyltyramine, or a pharmaceutically acceptable salt thereof, to the subject in an amount of 30 mg per kg per day to about 400 mg per kg per day for a period of time. The present disclosure also provides a method for maintaining weight of a subject or reducing diet induced weight gain of a subject and treating inflammation in a subject in need thereof, comprising administering N-trans caffeoyltyramine, or a pharmaceutically acceptable salt thereof, to the subject in an amount of 30 mg per kg per day to about 400 mg per kg per day for a period of time. In some embodiments, wherein the level of IL-6, TNFα, or nitric oxide in the subject is decreased.

In some embodiments, the HNF4α agonist is N-trans caffeoyltyramine (Compound A), or a pharmaceutically acceptable salt thereof, having a structure of:

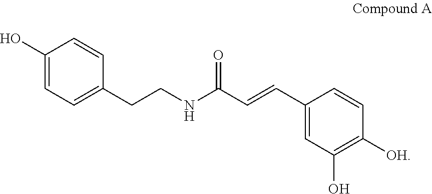 [SMILES: O=C(/C=C/c1ccc(O)c(O)c1)NCCc1ccc(O)cc1]

The present disclosure reveals that mice fed a high fat diet containing N-trans caffeoyltyramine (NCT) weighed substantially less than control mice fed high fat diet alone. The animals also had increased mitochondrial mass, exhibited increased fatty acid oxidation, and had an increased level of NAD. Markers of liver inflammation such as interleukin-6 (IL-6) and tumor necrosis factor alpha (TNFα), which are important in the progression of non-alcoholic fatty liver disease to non-alcoholic steatohepatitis were likewise decreased by NCT. There was no evidence of any toxicity from NCT consumption. These results show that HNF4α is an important regulator of mitochondrial mass and function and support the use of NCT as a means to treat, for example, disorders of aging, fatty acid excess, obesity, NAFLD, and NASH.

In some embodiments, wherein the N-trans caffeoyltyramine, or a pharmaceutically acceptable salt thereof, is administered orally, intravenously, intramuscularly, intraperitoneally, subcutaneously, or transdermally. In some embodiments, wherein the N-trans caffeoyltyramine, or a pharmaceutically acceptable salt thereof is administered orally. In some embodiments, wherein the N-trans caffeoyltyramine, or a pharmaceutically acceptable salt thereof, is administered once a day (q.d.), twice a day (b.i.d.), three times a day (t.i.d.), or four times a day (q.i.d.). In some embodiments, wherein the N-trans caffeoyltyramine, or a pharmaceutically acceptable salt thereof, is administered for at least 2, 3, 4, 5, 6, 7, 8, 9, 10, 11, or 12 weeks, or a period of time within a range defined by any of the preceding values. In some embodiments, wherein the N-trans caffeoyltyramine, or a pharmaceutically acceptable salt thereof, is administered for at least 1, 2, 3, 4, 5, 6, 7, 8, 9, 10, 11, or 12 months, or a period of time within a range defined by any of the preceding values. In some embodiments, wherein the subject is having a high fat diet (HFD). In some embodiments, wherein the subject has obesity. In some embodiments, wherein the subject has a BMI of at least about 25, about 26, about 27, about 28, about 29, about 30, or about 32. In some embodiments, wherein the body weight of the subject is reduced by at least about 10%, 20%, 30%, 40%, or 50%, or reduced an amount by weight % within a range defined by any of the preceding values, as compared to a reference subject having a similar body weight and the same diet but is not administered the N-trans caffeoyltyramine, or a pharmaceutically acceptable salt thereof. In some embodiments, wherein mitochondrial mass and/or function is increased in the subject. In some embodiments, wherein the mitochondrial mass/or function is measured by the expression level of cytochrome C or succinate dehydrogenase (SDHA). In some embodiments, wherein the mitochondrial mass and/or function is increased in the subject's liver. In some embodiments, wherein the mitochondrial mass and/or function is increased in primary hepatocytes of the subject. In some embodiments, wherein total cellular Nicotinamide Adenine Dinucleotide (NAD) level is increased in the subject. In some embodiments, further comprising measuring the expression level of VDAC1, citrate synthase, sirtuin, or PPARGC1A (PGC1a). In some embodiments, wherein an increase in expression level of VDAC1, citrate synthase, sirtuin, or PPARGC1A (PGC1a) as compared to a reference expression level is indicative of effectiveness of the N-trans caffeoyltyramine in reducing the body weight of the subject. In some embodiments, wherein the expression level of VDAC1, citrate synthase, sirtuin, or PPARGC1A (PGC1a) is measured in a sample from liver, intestine, pancreas, or kidney of the subject. In some embodiments, wherein the subject is a human. In some embodiments, the method comprises use of a composition comprising an effective amount of N-trans caffeoyltyramine, or a pharmaceutically acceptable salt thereof, for the treatment of one or more of the following conditions: aging, fatty acid excess, obesity, NAFLD, or NASH.

### BRIEF DESCRIPTION OF THE DRAWINGS

The patent application file contains at least one drawing executed in color. Copies of this patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows liver sections from mice fed normal chow (NC), high fat diet (HFD), or HFD+NCT immune stained for HNF4α or DAPI, described in Example 6. Scale bar=200 μM.

FIG. 1B shows the quantification of HNF4α fluorescence intensity from images stained as described in FIG. 1A, and further described in Example 6. Values represent the mean±SEM. *** p<0.001 (HFD vs. NC or HFD vs. HFD+NCT).

FIG. 1C depicts the body weight of mice measured each week for 10 weeks, as described in Example 6. Values represent the mean±SEM. * p<0.05, ** p<0.01 (HFD vs. HFD+NCT).

FIG. 1D depicts the consumption per cage of NC, HFD, or HFD+NCT measured each week for 10 weeks, as described in Example 6. Values represent the mean±SEM. NS non-significant (HFD vs. HFD+NCT).

FIG. 1E shows the stool triglyceride (TG) levels in mice, as described in Example 6. Values represent the mean±SEM. NS non-significant (HFD vs. HFD+NCT).

FIG. 1F shows images of subcutaneous fat in mice, as described in Example 6.

FIG. 1G shows images of livers in mice, as described in Example 6.

FIG. 1H depicts the quantification of liver weight in mice, as described in Example 6. Values represent the mean±SEM. *** p<0.001 (HFD vs. NC or HFD vs. HFD+NCT).

FIG. 1I shows the Oil Red O staining of livers in mice, as described in Example 6.

FIG. 1J depicts the quantification of the fold change in intensity of Oil Red O staining of livers in mice, as described in Example 6. Fold change was measured relative to NC. Values represent the mean±SEM. *** p<0.001 (HFD vs. NC or HFD vs. HFD+NCT).

FIG. 1K depicts the quantification of the stool triglyceride (TG) levels in mice, as described in Example 6. Values represent the mean±SEM. *** p<0.001 (HFD vs. NC or HFD vs. HFD+NCT).

FIG. 1L shows images of epididymal fat pad in mice, as described in Example 6.

FIG. 1M depicts the quantification of the epididymal fat pad weight in mice, as described in Example 6. Values represent the mean±SEM. *** p<0.001, NS non-significant (HFD vs. NC or HFD vs. HFD+NCT).

FIG. 1N shows images of subcutaneous fat in mice, as described in Example 6. Scale bar=200 μM.

FIG. 2A shows the quantification of mitochondrial fatty acid oxidation (FAO) activity, per μg of protein, in the presence of octanoyl CoA in mice, as described in Example 7. Dots indicate individual mice (N=6). Values represent the mean±SEM. * p<0.05 (HFD vs. HFD+NCT).

FIG. 2B shows the quantification of mitochondrial FAO activity, per μg of protein, in the absence of octanoyl CoA in mice, as described in Example 7. Dots indicate individual mice (N=6). Values represent the mean±SEM. ** p<0.01 (HFD vs. HFD+NCT).

FIG. 2C shows the quantification of mitochondrial FAO activity in mice, per μg of protein, as described in Example 7. Dots indicate individual mice (N=6). Values represent the mean #SEM. NS non-significant (HFD vs. HFD+NCT).

FIG. 2D shows the quantification of total cellular nicotinamide adenine dinucleotide (NAD) in mice per mg of protein, as described in Example 7. Dots indicate individual mice (N=6). Values represent the mean±SEM. ** p<0.01 (HFD vs. HFD+NCT).

FIG. 3A shows liver sections from mice fed normal chow (NC), high fat diet (HFD), or HFD+NCT immune stained for VDAC1 and DAPI nuclear stain. Scale bar=200 μM.

FIG. 3B shows the fold change in levels of VDAC1 in mouse livers normalized to NC. Values represent the mean±SEM. *** p<0.001 (HFD vs. NC or HFD vs. HFD+NCT).

FIG. 3C shows the levels of citrate synthase in mouse livers. Values represent the mean±SEM. *** p<0.001 (HFD vs. NC or HFD vs. HFD+NCT).

FIG. 3D shows gels probed for hepatic cytochrome C and SDHA protein from liver of mice fed normal chow (NC), high fat diet (HFD), or HFD+NCT.

FIG. 3E illustrates fold change in levels of hepatic cytochrome C in mouse livers normalized to NC. Values represent the mean±SEM. ** p<0.01 (HFD vs. NC or HFD vs. HFD+NCT).

FIG. 3F illustrates fold change in levels of SDHA in mouse livers normalized to NC. Values represent the mean±SEM. NS non-significant, ** p<0.01 (HFD vs. NC or HFD vs. HFD+NCT).

FIG. 3G shows cytochrome C mRNA levels in mouse livers. Values represent the mean±SEM. NS non-significant (HFD vs. NC or HFD vs. HFD+NCT).

FIG. 3H shows SDHA mRNA levels in mouse livers. Values represent the mean±SEM. NS non-significant and ** p<0.01 (HFD vs. NC or HFD vs. HFD+NCT).

FIG. 3I shows cytochrome C mRNA levels in primary human hepatocytes. Values represent the mean±SEM. NS non-significant (0 μM vs. each concentration of NCT in human hepatocyte).

FIG. 3J shows SDHA mRNA levels in primary human hepatocytes. Values represent the mean±SEM. * p<0.05, NS non-significant (0 μM vs. each concentration of NCT in human hepatocyte).

FIG. 3K shows ND1 mRNA levels in mouse livers. Values represent the mean±SEM. *p<0.05, ** p<0.01, *** p<0.001 (HFD vs. NC or HFD vs. HFD+NCT).

FIG. 3L shows 16s rRNA levels in mouse livers. Values represent the mean±SEM. *p<0.05, *** p<0.001 (HFD vs. NC or HFD vs. HFD+NCT).

FIG. 3M shows HSP60 mRNA levels in mouse livers. Values represent the mean±SEM (HFD vs. NC or HFD vs. HFD+NCT).

FIG. 3N shows PPARγ mRNA levels in mouse livers. Values represent the mean±SEM. *p<0.05, *** p<0.001 (HFD vs. NC or HFD vs. HFD+NCT).

FIG. 4A shows gels probed for PPARGC1A(PGC1a) protein from livers of mice fed normal chow (NC), high fat diet (HFD), or HFD+NCT.

FIG. 4B illustrates levels of fold change in PPARGC1A protein in mouse livers normalized to Ponceau S levels. Values represent the mean±SEM. ** p<0.01 (HFD vs. NC or HFD vs. HFD+NCT).

FIG. 4C shows PPARGC1A mRNA levels in mouse livers. Values represent the mean±SEM. NS non-significant, *p<0.05 (HFD vs. NC or HFD vs. HFD+NCT).

FIG. 4D shows Sirtuin1 mRNA levels in mouse livers. Values represent the mean±SEM. *p<0.05 (HFD vs. NC or HFD vs. HFD+NCT).

FIG. 4E shows Sirtuin3 mRNA levels in mouse livers. Values represent the mean±SEM. *p<0.05, ** p<0.01 (HFD vs. NC or HFD vs. HFD+NCT).

FIG. 4F shows PPARGC1A mRNA levels in primary human hepatocytes. Values represent the mean±SEM. * p<0.05, NS non-significant (0 μM vs. each concentration of NCT in human hepatocyte).

FIG. 4G shows Sirtuin1 mRNA levels in primary human hepatocytes. Values represent the mean±SEM. * p<0.05, NS non-significant (0 μM vs. each concentration of NCT in human hepatocyte).

FIG. 4H shows Sirtuin3 mRNA levels in primary human hepatocytes. Values represent the mean±SEM. * p<0.05, NS non-significant (0 μM vs. each concentration of NCT in human hepatocyte).

FIG. 5A shows IL-6 mRNA levels in mouse livers. Dots indicate individual mouse donors. Values represent the mean±SEM. NS non-significant, *p<0.05 (HFD vs. NC or HFD vs. HFD+NCT).

FIG. 5B shows TNFα mRNA levels in mouse livers. Dots indicate individual mouse donors. Values represent the mean±SEM. NS non-significant, *p<0.05 (HFD vs. NC or HFD vs. HFD+NCT).

FIG. 5C shows ILIβ mRNA levels in mouse livers. Dots indicate individual mouse or human donors. Values represent the mean±SEM. NS non-significant (HFD vs. NC or HFD vs. HFD+NCT).

FIG. 5D illustrates levels of IL-6 protein secreted by primary human hepatocytes. Dots indicate individual human donors. Values represent the mean±SEM. * p<0.05, NS non-significant (0 μM vs. each concentration of NCT in primary human hepatocytes).

FIG. 5E shows IL-6 mRNA levels in primary human hepatocytes. Dots indicate individual human donors. Values represent the mean±SEM. * p<0.05, NS non-significant (0 μM vs. each concentration of NCT in primary human hepatocytes).

FIG. 5F shows TNFα mRNA levels in primary human hepatocytes. Dots indicate individual human donors. Values represent the mean±SEM. * p<0.05, NS non-significant (0 μM vs. each concentration of NCT in primary human hepatocytes).

FIG. 5G shows the levels of nitric oxide (NO) in the livers of mice fed on HFD. Dots indicate individual mouse. Values represent the mean±SEM. ** p<0.01, *** p<0.001 (HFD vs. NC or HFD vs. HFD+NCT).

FIG. 5H shows the levels of NO in cultured T6PNE cells treated with palmitate. Dots indicate biological replicates. Values represent the mean±SEM. * p<0.05, ** p<0.01 (DMSO vs. DMSO+Palmitate or DMSO+Palmitate vs. NCT+Palmitate).

FIG. 5I shows the blood alanine transaminase (ALT) levels in mice fed on HFD. Dots indicate individual mouse donors. Values represent the mean±SEM. * p<0.05, ** p<0.01 (HFD vs. NC or HFD vs. HFD+NCT).

### INCORPORATION BY REFERENCE

All publications, patents, and patent applications mentioned in this specification are herein incorporated by reference to the same extent as if each individual publication, patent, or patent application was specifically and individually indicated to be incorporated by reference. For example, Veeriah, V., Lee, SH. & Levine, F. Long-term oral administration of an HNF4α agonist prevents weight gain and hepatic steatosis by promoting increased mitochondrial mass and function. Cell Death Dis 13, 89 (2022) (doi.org/10.1038/s41419-022-04521-5) is incorporated herein by reference in its entirety. To the extent publications and patents or patent applications incorporated by reference contradict the disclosure contained in the specification, the specification is intended to supersede and/or take precedence over any such contradictory material.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the present disclosure. It should be understood that various alternatives to the embodiments of the present disclosure described herein may be employed.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Oxo” refers to the ═O radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond. “Halo” or “halogen” refers to bromo, chloro, fluoro or iodo substituents.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, sodium, potassium, calcium, magnesium salts and the like.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### HNF4α Agonists

HNF4α is a nuclear receptor transcription factor that is expressed predominantly in the liver, intestine, pancreas, and kidney. In the liver, it plays an important role in metabolic homeostasis, including gluconeogenesis the urea cycle, and lipid metabolism. Natural ligand and antagonists of HNF4α include a subset of fatty acids, however, they are thought to play a structural rather than regulatory role, because the bound fatty acids are not exchangeable in the context of the ligand binding domain constructs. More recently, linoleic acid is shown to bind to HNF4α and to be exchangeable in vivo.

Agonists of HNF4α are screened out in a cell-based assay in which green fluorescent protein (GFP) is expressed under the control of the human insulin promoter, goal of finding compounds that might be relevant to metabolic syndrome and type 2 diabetes. Two drug molecules Alverine and Benfluorex are found to be weak agonists of HNF4α for distinct indications. Benfluorex was used to treat type 2 diabetes until its withdrawal because of side effects but did not have a clearly established mode of action. Alverine was used in the treatment of irritable bowel syndrome.

Recently, some agonists, including N-trans caffeoyltyramine (NCT) and N-trans feruloyltyramine (NFT), are found to be much more potent activators of HNF4α. They interact directly and so are true agonists and exhibit specificity for HNF4α, as HNF4α siRNA ablates their effect. Both NCT and NFT are found in plants, including some consumed by humans. They have no known role as nuclear receptor ligands or mediators of plant metabolism, and there is no HNF4α homolog in plants. Given their poor oral bioavailability and low abundance in plants that are commonly consumed as food, NCT and NFT are unlikely to be physiologically relevant sources of HNF4α ligands in most human diets.

It has been shown that NCT could reduce fat stored in liver of mice maintained on a 60% fat calorie diet, when applied through intraperitoneal injection for 2 weeks. NCT induced decreased hepatic steatosis due to stimulation of lipophagy. However, no body weight loss is observed.

### Compounds of HNF4α Agonists

In one aspect, provided herein is a HNF4α agonist compound.

One embodiment provides a compound, having the structure of Formula (I):

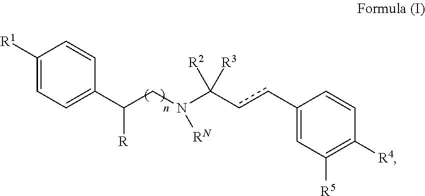

or pharmaceutically acceptable salt thereof,
wherein:
each of R¹, R⁴, and R⁵ is independently hydrogen, —OH, or —OCH₃;
R² and R³ are each hydrogen or taken together to form an oxo;
R is hydrogen, —OH, or halo;
R^{N} is hydrogen or C₁₋₃ alkyl; and
n is 0, 1, 2, or 3; and [symbol] is a single or double bond.

In some embodiments, R¹ and R⁴ are —OH, R² and R³ taken together to form an oxo, R^{N} is hydrogen, and n is 1.

In some embodiments, R¹, R², R³, R⁴, R⁵, and R are each hydrogen, R^{N} is ethyl, and n is 2.

In some embodiments, the HNF4α agonist is N-trans caffeoyltyramine (Compound A), having a structure of:

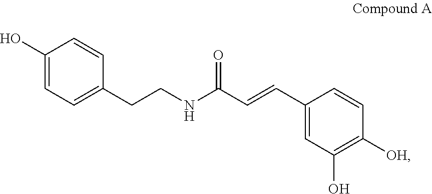 [SMILES: O=C(/C=C/c1ccc(O)c(O)c1)NCCc1ccc(O)cc1]

or pharmaceutically acceptable salt thereof.

### Preparation of Compounds

The compounds described herein are commercially available or made according to organic synthesis techniques known to those skilled in this art, starting from commercially available chemicals and/or from compounds described in the chemical literature.

Synthetic routes to prepare the compounds described herein can be developed using the Reaxys® Predictive Retrosynthesis tool.

Specific and analogous reactants are optionally identified through the indices of known chemicals prepared by the Chemical Abstract Service of the American Chemical Society or Reaxys® of Elsevier, which are available in most public and university libraries, as well as through on-line databases. Chemicals that are known but not commercially available in catalogs are optionally prepared by custom chemical synthesis houses, where many of the standard chemical supply houses (e.g., those listed above) provide custom synthesis services.

### Pharmaceutical Compositions

In certain embodiments, the HNF4α agonist described herein (e.g., Compound A) is administered as a pure chemical. In other embodiments, the HNF4α agonist described herein (e.g., Compound A) is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one HNF4α agonist described herein (e.g., Compound A), pharmaceutically acceptable salt thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt thereof.

One embodiment provides a method of preparing a pharmaceutical composition comprising mixing a compound of Formula (I), or a pharmaceutically acceptable salt thereof, and a pharmaceutically acceptable carrier.

In certain embodiments, the HNF4α agonist as described by Formula (I), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and Compound A or a pharmaceutically acceptable salt thereof.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 2181 Ed. Mack Pub. Co., Easton, PA (2005)).

### Oral Administration

The pharmaceutical compositions provided herein for oral administration can be provided in solid, semisolid, or liquid dosage forms for oral administration. As used herein, oral administration also includes buccal, lingual, and sublingual administration.

Binders or granulators impart cohesiveness to a tablet to ensure the tablet remaining intact after compression. Suitable binders or granulators include, but are not limited to, starches, such as corn starch, potato starch, and pre-gelatinized starch. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; and mixtures thereof. The pharmaceutical compositions provided herein may contain about 0.1 to about 5% by weight of a lubricant.

Suitable glidants include, but are not limited to, colloidal silicon dioxide, and asbestos-free talc. Suitable coloring agents include, but are not limited to, any of the approved, certified, water soluble FD&C dyes, and water insoluble FD&C dyes suspended on alumina hydrate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate.

The pharmaceutical compositions provided herein for oral administration can be provided in liquid and semisolid dosage forms, including emulsions, solutions, suspensions, elixirs, and syrups.

Other useful liquid and semisolid dosage forms include, but are not limited to, those containing the active ingredient(s) provided herein, and a dialkylated mono- or poly-alkylene glycol, including, 1,2-dimetboxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT).

The pharmaceutical compositions provided herein for oral administration can be also provided in the forms of liposomes, micelles, microspheres, or nanosystems.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### Transdermal Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect.

Pharmaceutically acceptable carriers and excipients suitable for use in the topical formulations provided herein include, but are not limited to, aqueous vehicles, water-miscible vehicles, and non-aqueous vehicles.

The pharmaceutical compositions can also be administered topically by electroporation, iontophoresis, phonophoresis, sonophoresis, or microneedle or needle-free injection.

The pharmaceutical compositions provided herein can be provided in the forms of ointments, creams, and gels.

Suitable cream base can be oil-in-water or water-in-oil. Suitable cream vehicles may be water-washable, and contain an oil phase, an emulsifier, and an aqueous phase.

Gels are semisolid, suspension-type systems. Single-phase gels contain organic macromolecules distributed substantially uniformly throughout the liquid carrier.

In some embodiments, the HNF4α agonist described herein (e.g., Compound A), or pharmaceutically acceptable salt thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one HNF4α agonist described herein (e.g., Compound A) differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the subject or patient.

### Methods of Use

One embodiment provides a compound of Formula (I), or a pharmaceutically acceptable salt thereof, for use in a method of treatment of the human or animal body.

One embodiment provides a method for reducing body weight of a subject, comprising administering to the subject a compound of Formula (I) or a pharmaceutically acceptable salt thereof. In certain embodiments, a therapeutically effective amount is administered.

One embodiment provides a compound of Formula (I), or a pharmaceutically acceptable salt thereof, for use in a method for reducing body weight of a subject.

One embodiment provides a pharmaceutical composition comprising a compound of Formula (I), or a pharmaceutically acceptable salt thereof, and a pharmaceutically acceptable excipient for use in a method for reducing body weight of a subject.

One embodiment provides a use of a compound of Formula (I), or a pharmaceutically acceptable salt thereof, in the manufacture of a medicament for reducing body weight of a subject.

One embodiment provides a method for reducing body weight of a subject, comprising administering to the subject Compound A or a pharmaceutically acceptable salt thereof. In certain embodiments, a therapeutically effective amount is administered.

One embodiment provides Compound A, or a pharmaceutically acceptable salt thereof, for use in a method for reducing body weight of a subject.

One embodiment provides a pharmaceutical composition comprising Compound A, or a pharmaceutically acceptable salt or solvate thereof, and a pharmaceutically acceptable excipient for use in a method for reducing body weight of a subject.

One embodiment provides a use of Compound A, or a pharmaceutically acceptable salt thereof, in the manufacture of a medicament for reducing body weight of a subject.

BMI (body mass index) is a key sign of overall health. Guidelines recommend that all adults keep their BMI between 18 and 24.9. A BMI of 25 and over indicates that you are overweight. And a BMI over 30 is considered obesity. BMI is still widely used in the medical community because it's an inexpensive and quick way to analyze a person's potential health status and outcomes.

In some embodiments, a compound of Formula (I), or a pharmaceutically acceptable salt thereof, may be administered to a subject with overweight or obesity. In some embodiments, a compound of Formula (I), or a pharmaceutically acceptable salt thereof, may be administered to a subject with BMI that is about 25 to about 65. In some embodiments, a compound of Formula (I), or a pharmaceutically acceptable salt thereof, may be administered to a subject with BMI that is about 25 to about 26, about 25 to about 27, about 25 to about 28, about 25 to about 29, about 25 to about 30, about 25 to about 32, about 25 to about 35, about 25 to about 40, about 25 to about 45, about 25 to about 50, about 25 to about 55, about 25 to about 60, about 25 to about 65, about 26 to about 27, about 26 to about 28, about 26 to about 29, about 26 to about 30, about 26 to about 32, about 26 to about 35, about 26 to about 40, about 26 to about 45, about 26 to about 50, about 26 to about 55, about 26 to about 60, about 26 to about 65, about 27 to about 28, about 27 to about 29, about 27 to about 30, about 27 to about 32, about 27 to about 35, about 27 to about 40, about 27 to about 45, about 27 to about 50, about 27 to about 55, about 27 to about 60, about 27 to about 65, about 28 to about 29, about 28 to about 30, about 28 to about 32, about 28 to about 35, about 28 to about 40, about 28 to about 45, about 28 to about 50, about 28 to about 55, about 28 to about 60, about 28 to about 65, about 29 to about 30, about 29 to about 32, about 29 to about 35, about 29 to about 40, about 29 to about 45, about 29 to about 50, about 29 to about 55, about 29 to about 60, about 29 to about 65, about 30 to about 32, about 30 to about 35, about 30 to about 40, about 30 to about 45, about 30 to about 50, about 30 to about 55, about 30 to about 60, about 30 to about 65, about 32 to about 35, about 32 to about 40, about 32 to about 45, about 32 to about 50, about 32 to about 55, about 32 to about 60, about 32 to about 65, about 35 to about 40, about 35 to about 45, about 35 to about 50, about 35 to about 55, about 35 to about 60, about 35 to about 65, about 40 to about 45, about 40 to about 50, about 40 to about 55, about 40 to about 60, about 40 to about 65, about 45 to about 50, about 45 to about 55, about 45 to about 60, about 45 to about 65, about 50 to about 55, about 50 to about 60, about 50 to about 65, about 55 to about 60, about 55 to about 65, or about 60 to about 65. In some embodiments, a compound of Formula (I), or a pharmaceutically acceptable salt thereof, may be administered to a subject with BMI that is at least about 25, at least about 26, at least about 27, at least about 28, at least about 29, at least about 30, at least about 32, at least about 35, at least about 40, at least about 45, at least about 50, at least about 55, at least about 60, at least about 65. In some embodiments, the compound of Formula (I), or a pharmaceutically acceptable salt thereof, is Compound A, or a pharmaceutically acceptable salt thereof.

In some embodiments, a compound of Formula (I), or a pharmaceutically acceptable salt thereof, may be administered to a subject with overweight or obesity to reduce body weight. In some embodiments, the body weight of the subject may be reduced by about 5% to about 80%, as compared to a reference subject having a similar body weight and the same diet but is not administered the compound of Formula (I), or a pharmaceutically acceptable salt thereof. In some embodiments, the body weight of the subject may be reduced by about 5% to about 10%, about 5% to about 20%, about 5% to about 30%, about 5% to about 40%, about 5% to about 50%, about 5% to about 60%, about 5% to about 70%, about 5% to about 80%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80%, as compared to a reference subject having a similar body weight and the same diet but is not administered the compound of Formula (I), or a pharmaceutically acceptable salt thereof. In some embodiments, the body weight of the subject may be reduced by about 5%, about 10%, about 20%, about 30%, about 40%, about 50%, about 60%, about 70%, or about 80%, as compared to a reference subject having a similar body weight and the same diet but is not administered the compound of Formula (I), or a pharmaceutically acceptable salt thereof. In some embodiments, the body weight of the subject may be reduced by at least about 5%, at least about 10%, at least about 20%, at least about 30%, at least about 40%, at least about 50%, at least about 60%, at least about 70%, or at least about 80%, as compared to a reference subject having a similar body weight and the same diet but is not administered the compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

A high fat diet (HFD) is a diet wherein at least 35% of total calories is consumed from fats, both unsaturated and saturated. In addition to the popular processed foods, many other foods have a high fat content including but not limited to animal fat, chocolate, butter, and oily fish. In some embodiments, a compound of Formula (I), or a pharmaceutically acceptable salt thereof, may be administered to a subject having a high fat diet. In some embodiments, the compound of Formula (I), or a pharmaceutically acceptable salt thereof, is Compound A, or a pharmaceutically acceptable salt thereof.

Overweight and obesity increase the risk for many health problems, such as type 2 diabetes, high blood pressure, heart disease, stroke, joint problems, liver disease, gallstones, some types of cancer, and sleep and breathing problems, kidney disease, as well as gestational diabetes, preeclampsia (high blood pressure) during pregnancy.

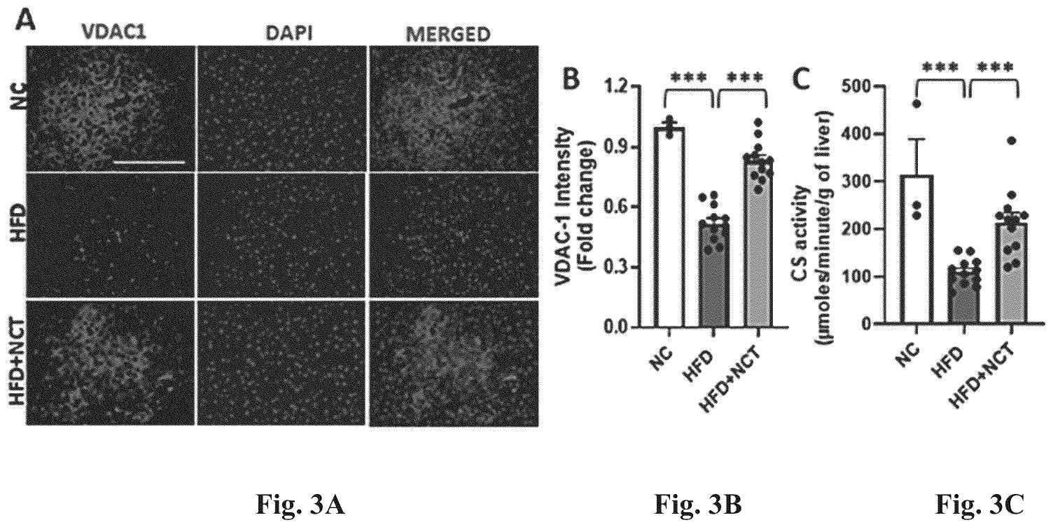

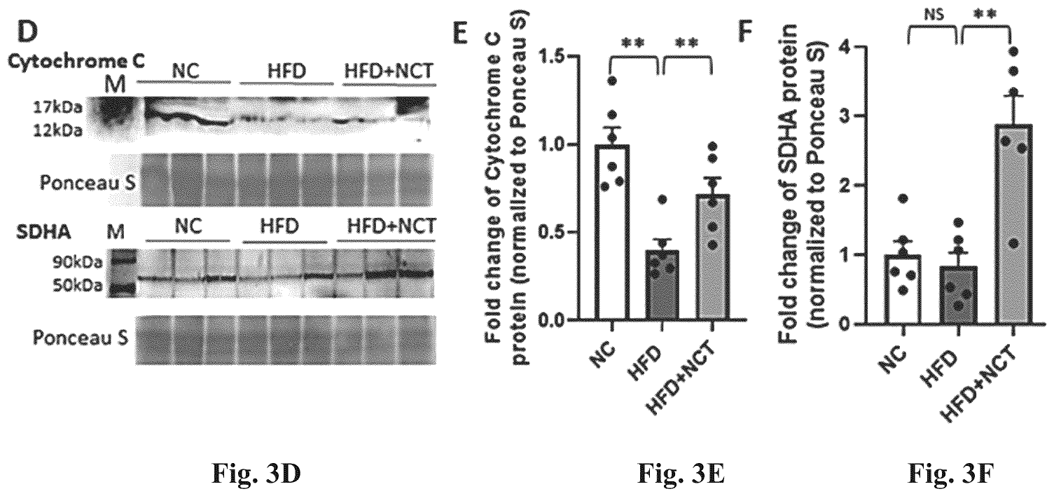

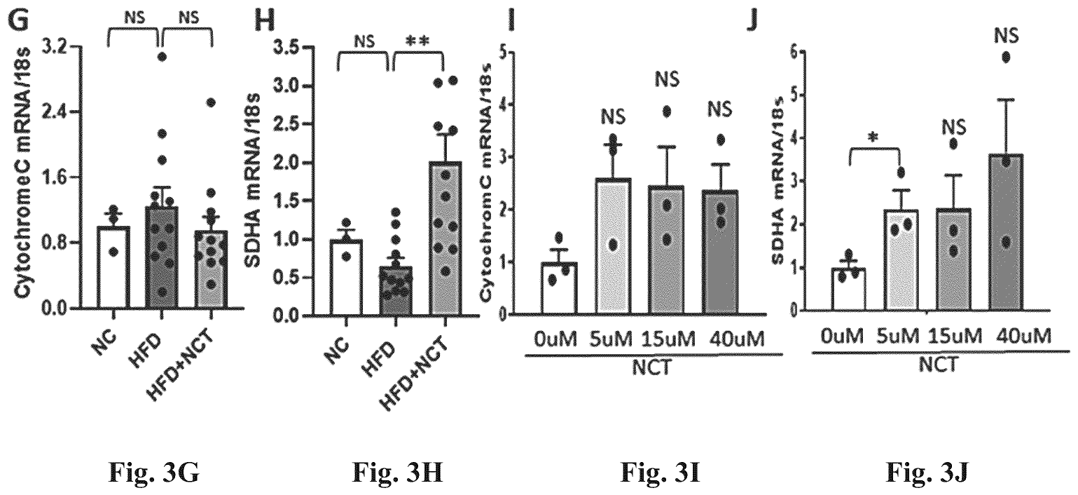

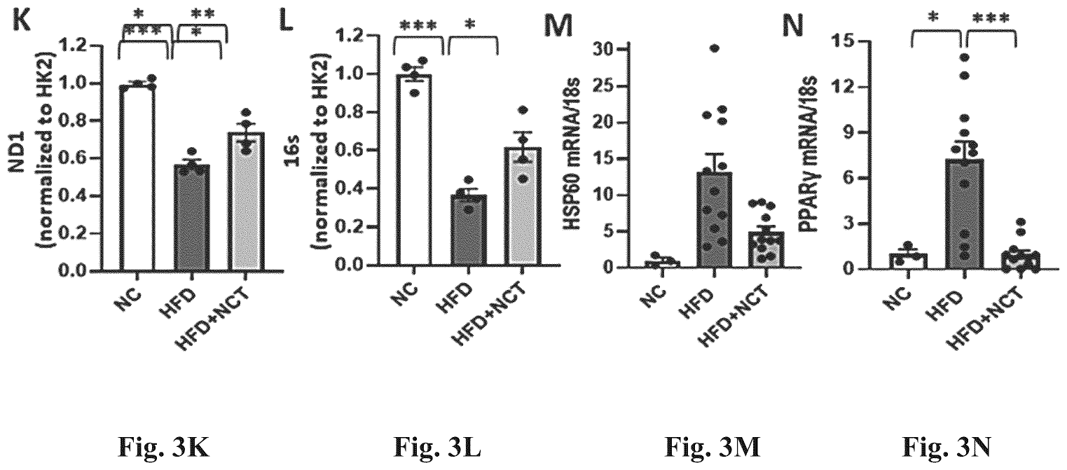

The major function of mitochondria is oxidative phosphorylation. Increased expression of proteins involved in oxidative phosphorylation may cause increasing mitochondrial mass. Cytochrome C and succinate dehydrogenase expression, both of which are important components of the respiratory chain. Cytochrome C plays a dual role: in mitochondria, being critical for mitochondrial respiration but also playing a role in cell survival. Succinate dehydrogenase, encoded by the SDHA gene, is the catalytic subunit of succinate-ubiquinone oxidoreductase, a complex of the mitochondrial respiratory chain. HFD decreased hepatic cytochrome C and SDHA protein levels. In some embodiments, increased expression level of cytochrome C or SDHA may be indicative of increased mitochondrial mass and/or function. In some embodiments, the mitochondrial mass and/or function may be increased in the subject's liver. In some embodiments, the mitochondrial mass and/or function may be increased in primary hepatocytes of the subject. In some embodiments, administering a compound of Formula (I), or a pharmaceutically acceptable salt thereof, to a subject, may reverse the depression on levels of hepatic cytochrome C and SDHA protein caused by HFD, as illustrated in FIGS. 3D-3F for administering Compound A. In some embodiments, administering a compound of Formula (I), or a pharmaceutically acceptable salt thereof, to a subject, may increase SDHA mRNA in mouse liver, as illustrated in FIGS. 3G and 3H for administering Compound A. In some embodiments, administering a compound of Formula (I), or a pharmaceutically acceptable salt thereof, to a subject, may increase SDHA mRNA in human hepatocytes, as illustrated in FIGS. 3I and 3J for administering Compound A. In some embodiments, the method described herein, further comprises measuring the expression level of SDHA mRNA. In some further embodiments, the expression level of SDHA mRNA may be measured in a sample from liver, intestine, pancreas, or kidney of the subject. In some embodiments, the increased expression level of SDHA mRNA may be indicative of effectiveness of administering a compound of Formula (I), or a pharmaceutically acceptable salt thereof, in reducing the body weight of the subject. In some embodiments, administering a compound of Formula (I), or a pharmaceutically acceptable salt thereof, to a subject, may increase mitochondrial mass and/or function. In some embodiments, administering a compound of Formula (I), or a pharmaceutically acceptable salt thereof, to a subject, may induce an increase in mitochondrial DNA. In some embodiments, administering a compound of Formula(I), or a pharmaceutically acceptable salt thereof, to a subject, may increase the level of DNA encoding the mitochondrial genes ND1. In some embodiments, administering a compound of Formula(I), or a pharmaceutically acceptable salt thereof, to a subject, may increase the level of DNA encoding the mitochondrial genes 16s rRNA, as illustrated in FIGS. 3K and 3L for administering Compound A. In some embodiments, the compound of Formula (I), or a pharmaceutically acceptable salt thereof, is Compound A, or a pharmaceutically acceptable salt thereof.

In some embodiments, administering a compound of Formula (I), or a pharmaceutically acceptable salt thereof, to a subject, the increased mitochondrial mass and/or function may increase total fatty acid oxidation (FAO). In some embodiments, administering a compound of Formula (I), or a pharmaceutically acceptable salt thereof, to a subject, the increased mitochondrial mass and/or function may not increase the level of FAO per unit of mitochondrial mass. In some embodiments, administering a compound of Formula (I), or a pharmaceutically acceptable salt thereof, to a subject, the increased mitochondrial mass and/or function may increase total cellular nicotinamide adenine dinucleotide (NAD) level. In some embodiments, the method described herein, further comprises measuring the expression level of NAD. In some further embodiments, the expression level of NAD may be measured in a sample from liver, intestine, pancreas, or kidney of the subject. In some embodiments, the increased expression level of NAD may be indicative of effectiveness of administering a compound of Formula (I), or a pharmaceutically acceptable salt thereof in reducing the body weight of the subject. In some embodiments, administering a compound of Formula (I), or a pharmaceutically acceptable salt thereof, to a subject, the increased FAO may be a secondary effect. In some embodiments, the compound of Formula (I), or a pharmaceutically acceptable salt thereof, is Compound A, or a pharmaceutically acceptable salt thereof.

Voltage-dependent anion channel 1 (VDAC1) and citrate synthase are two commonly used protein markers of mitochondrial mass. VDAC1 is located on the outer mitochondrial membrane, and citrate synthase is located in the mitochondrial matrix. In some embodiments, administering a compound of Formula (I), or a pharmaceutically acceptable salt thereof, to a subject, may reverse the depression on levels of VDAC1 and citrate synthase caused by HFD, as illustrated in FIGS. 3A-3C for administering Compound A. In some embodiments, the method described herein, further comprises measuring the expression level of VDAC1. In some further embodiments, the expression level of VDAC1 may be measured in a sample from liver, intestine, pancreas, or kidney of the subject. In some embodiments, the increased expression level of VDAC1 may be indicative of effectiveness of administering a compound of Formula (I), or a pharmaceutically acceptable salt thereof in reducing the body weight of the subject. In some embodiments, the method described herein, further comprises measuring the expression level of citrate synthase. In some further embodiments, the expression level of citrate synthase may be measured in a sample from liver, intestine, pancreas, or kidney of the subject. In some embodiments, the increased expression level of citrate synthase may be indicative of effectiveness of administering a compound of Formula (I), or a pharmaceutically acceptable salt thereof in reducing the body weight of the subject. In some embodiments, the compound of Formula (I), or a pharmaceutically acceptable salt thereof, is Compound A, or a pharmaceutically acceptable salt thereof.

Mitochondrial stress is an important feature of NAFLD and its progression to NASH. HSP60 is a mitochondrial chaperone and is induced by mitochondrial stress. In some embodiments, administering a compound of Formula (I), or a pharmaceutically acceptable salt thereof, to a subject, may reverse the induction of levels of HSP60 mRNA caused by HFD, as illustrated in FIG. 3M for administering Compound A. In some embodiments, the method described herein, further comprises measuring the expression level of HSP60 mRNA. In some further embodiments, the expression level of HSP60 mRNA may be measured in a sample from liver, intestine, pancreas, or kidney of the subject. In some embodiments, the increased expression level of HSP60 mRNA may be indicative of effectiveness of administering a compound of Formula (I), or a pharmaceutically acceptable salt thereof in reducing the body weight of the subject. In some embodiments, the compound of Formula (I), or a pharmaceutically acceptable salt thereof, is Compound A, or a pharmaceutically acceptable salt thereof.

PPARγ plays an important role in the mitochondrial stress response. It is activated by fatty acids and exhibits high expression in fatty liver disease. In some embodiments, administering a compound of Formula (I), or a pharmaceutically acceptable salt thereof, to a subject, may reduce PPARγ expression to the level in the livers of mice on normal chow, as illustrated in FIG. 3N for administering Compound A. In some embodiments, the method described herein, further comprises measuring the expression level of PPARy. In some further embodiments, the expression level of PPARγ may be measured in a sample from liver, intestine, pancreas, or kidney of the subject. In some embodiments, the increased expression level of PPARγ may be indicative of effectiveness of administering a compound of Formula (I), or a pharmaceutically acceptable salt thereof in reducing the body weight of the subject. In some embodiments, the compound of Formula (I), or a pharmaceutically acceptable salt thereof, is Compound A, or a pharmaceutically acceptable salt thereof.

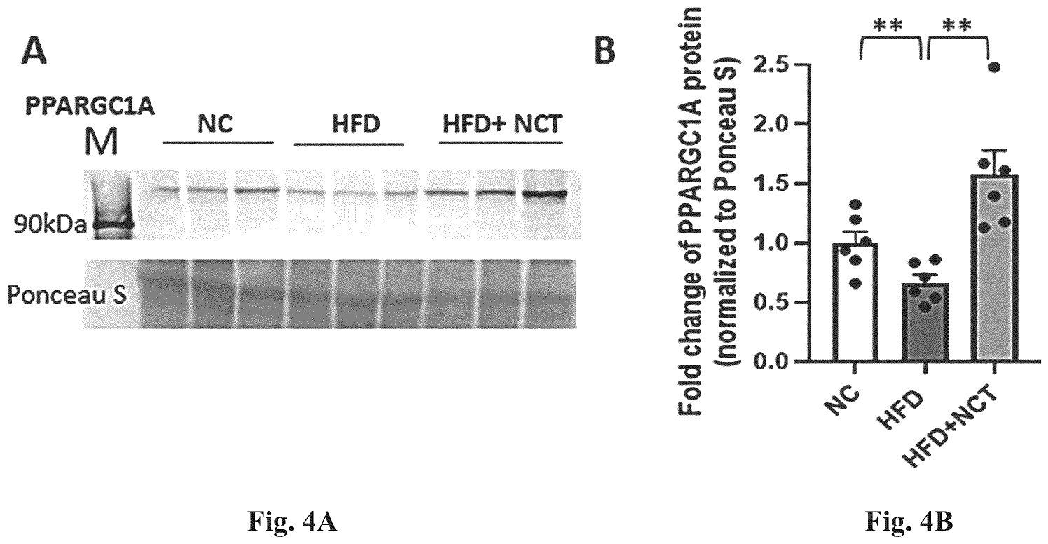

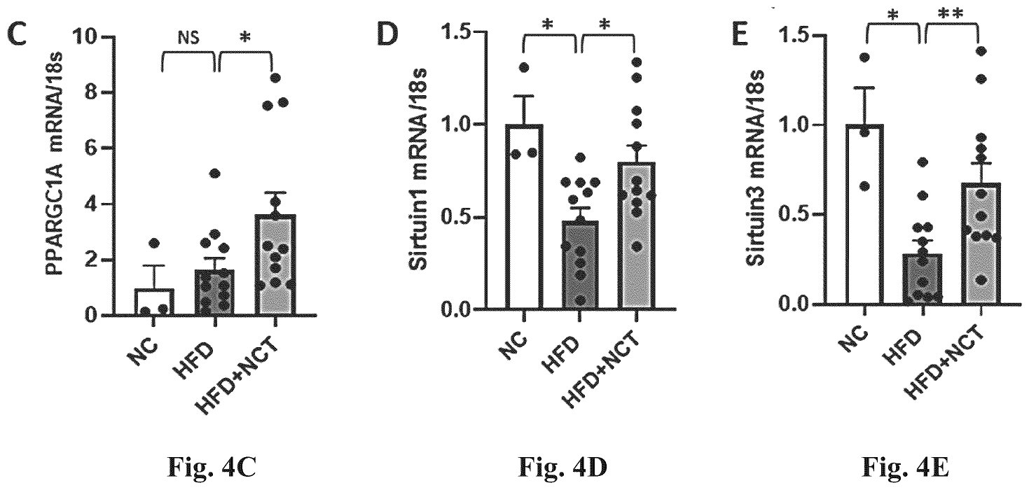

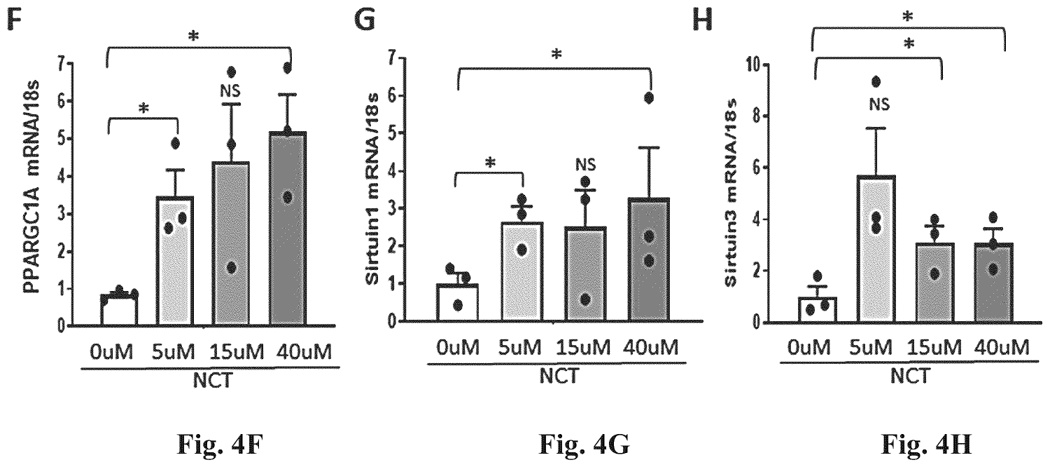

PPARGC1A (PGC1a) plays an important role in mitochondrial biogenesis but is not known to be regulated by HNF4α. In some embodiments, administering a compound of Formula (I), or a pharmaceutically acceptable salt thereof, to a subject, may increase PPARGC1A mRNA by multiple folds (e.g. 6 folds). In some embodiments, administering a compound of Formula (I), or a pharmaceutically acceptable salt thereof, to a subject, may increase PPARGC1A(PGC1a) protein and mRNA levels in mouse liver, as illustrated in FIGS. 4A-4C for administering Compound A. In some embodiments, administering a compound of Formula (I), or a pharmaceutically acceptable salt thereof, to a subject, may increase PPARGC1A(PGC1a) mRNA in primary human hepatocytes, as illustrated in FIG. 4F for administering Compound A. In some embodiments, the method described herein, further comprises measuring the expression level of PPARGC(PGC1a). In some further embodiments, the expression level of PPARGC(PGC1a) may be measured in a sample from liver, intestine, pancreas, or kidney of the subject. In some embodiments, the increased expression level of PPARGC(PGC1a) may be indicative of effectiveness of administering a compound of Formula (I), or a pharmaceutically acceptable salt thereof in reducing the body weight of the subject. In some embodiments, the compound of Formula (I), or a pharmaceutically acceptable salt thereof, is Compound A, or a pharmaceutically acceptable salt thereof.

Activity of PPARGC1A is controlled by sirtuins, which are NAD-dependent deacetylases. They are both downstream targets of transcriptional activation by PPARGC1A, and activators of PPARGC1A activity through deacetylation. In some embodiments, administering Formula (I), or a pharmaceutically acceptable salt thereof, to a subject, may reverse the depression on levels of Sirt1 mRNA in mouse liver caused by HFD, as illustrated in FIG. 4D for administering Compound A, or a pharmaceutically acceptable salt thereof. In some embodiments, administering Formula (I), or a pharmaceutically acceptable salt thereof, to a subject, may reverse the depression on levels of Sirt3 mRNA in mouse liver caused by HFD, as illustrated in FIG. 4E for administering Compound A, or a pharmaceutically acceptable salt thereof. In some embodiments, administering Formula (I), or a pharmaceutically acceptable salt thereof, to a subject, may reverse the depression on levels Sirt1 mRNA in primary human hepatocytes caused by HFD, as illustrated in FIG. 4G for administering Compound A, or a pharmaceutically acceptable salt thereof. In some embodiments, administering Formula (I), or a pharmaceutically acceptable salt thereof, to a subject, may reverse the depression on levels Sirt3 mRNA in primary human hepatocytes caused by HFD, as illustrated in FIG. 4H for administering Compound A, or a pharmaceutically acceptable salt thereof. In some embodiments, the method described herein, further comprises measuring the expression level of Sirt1 mRNA. In some further embodiments, the expression level of Sirt1 mRNA may be measured in a sample from liver, intestine, pancreas, or kidney of the subject. In some embodiments, the increased expression level of Sirt1 mRNA may be indicative of effectiveness of administering a compound of Formula (I), or a pharmaceutically acceptable salt thereof, in reducing the body weight of the subject. In some embodiments, the method described herein, further comprises measuring the expression level of Sirt3 mRNA. In some further embodiments, the expression level of Sirt3 mRNA may be measured in a sample from liver, intestine, pancreas, or kidney of the subject. In some embodiments, the increased expression level of Sirt3 mRNA may be indicative of effectiveness of administering a compound of Formula (I), or a pharmaceutically acceptable salt thereof in reducing the body weight of the subject. Poly(ADP-ribose) polymerases (PARPs) inhibit mitochondrial function and PPARGC1A activity. In some embodiments, administering a compound of Formula (I), or a pharmaceutically acceptable salt thereof, to a subject, may not affect expression levels of Parp1 or Parp2 mRNA. In some embodiments, the compound of Formula (I), or a pharmaceutically acceptable salt thereof, is Compound A, or a pharmaceutically acceptable salt thereof.

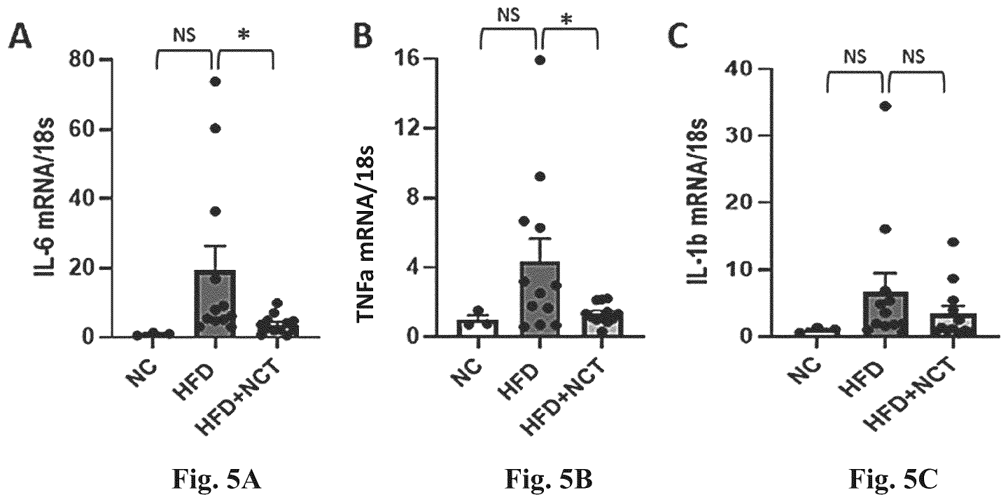

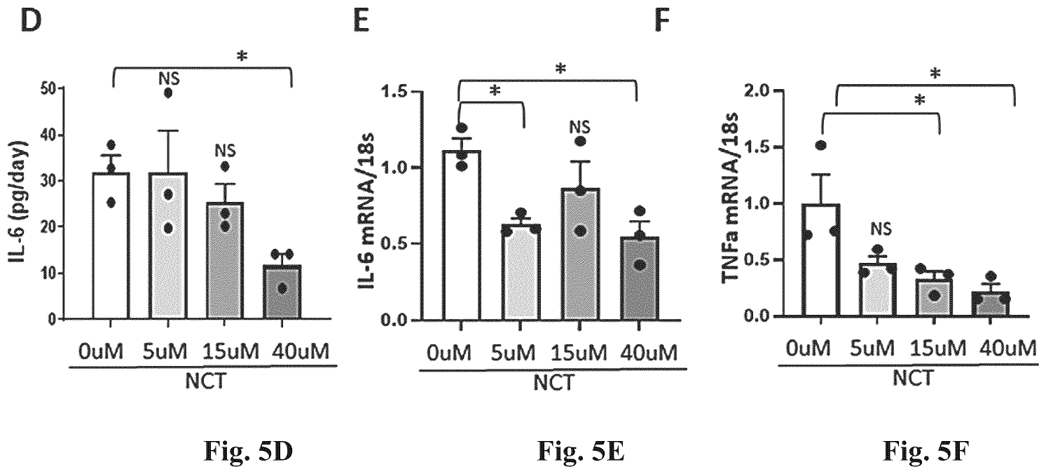

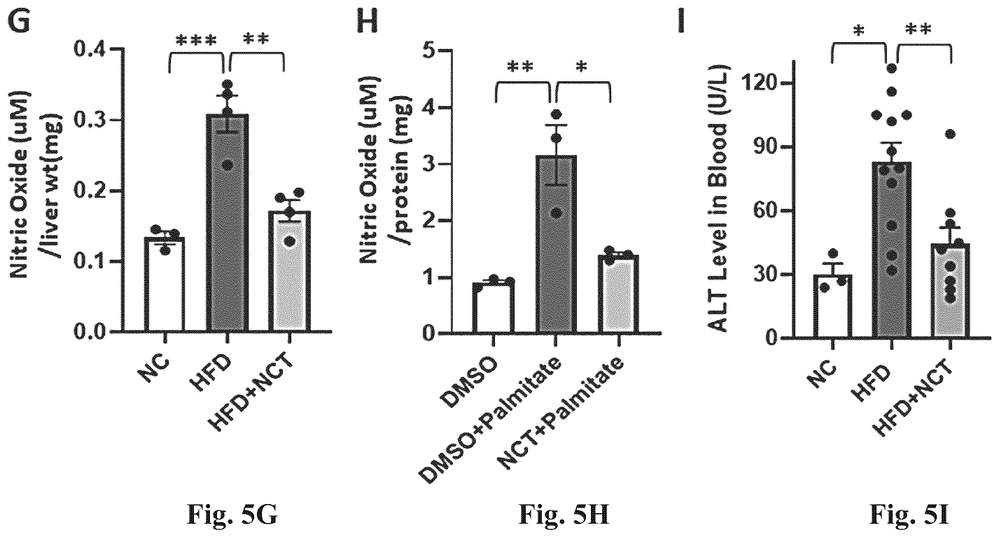

Inflammation is a major factor in the pathophysiology of NAFLD and its progression to NASH. Inflammation contributes to essential features of NASH pathology, including fibrosis and hepatocyte death, ultimately leading to cirrhosis. IL-6 and TNFα are inflammatory mediators that are important in NASH. In some embodiments, administering a compound of Formula (I), or a pharmaceutically acceptable salt thereof, to a subject, may decrease IL-6 level in livers of mice on HFD, as illustrated in FIG. 5A for administering Compound A. In some embodiments, administering a compound of Formula (I), or a pharmaceutically acceptable salt thereof, to a subject, may decrease TNFα level in livers of mice on HFD, as illustrated in FIG. 5B for administering Compound A. In some embodiments, administering a compound of Formula (I), or a pharmaceutically acceptable salt thereof, to a subject, may decrease IL-6 mRNA and protein levels in cultured human hepatocytes, as illustrated in FIGS. 5D and 5E for administering Compound A. In some embodiments, administering a compound of Formula (I), or a pharmaceutically acceptable salt thereof, to a subject, may decrease TNFα mRNA level in a dose-responsive manner in cultured human hepatocytes, as illustrated in FIG. 5F for administering Compound A. In some embodiments, administering a compound of Formula (I), or a pharmaceutically acceptable salt thereof, to a subject, may not affect the expression level of ILIβ, as illustrated in FIG. 5C for administering Compound A. In some embodiments, the compound of Formula (I), or a pharmaceutically acceptable salt thereof, is Compound A, or a pharmaceutically acceptable salt thereof.

Nitric oxide (NO) plays an important role in inflammatory responses, including NAFLD. NO is increased in the livers of mice fed on HFD, as illustrated in FIG. 5G; as well as in cultured cells treated with palmitate, as illustrated in FIG. 5H. In some embodiments, administering a compound of Formula (I), or a pharmaceutically acceptable salt thereof, to a subject, may decrease the level of NO in the livers of mice fed on HFD, as illustrated in FIG. 5G for administering Compound A. In some embodiments, administering a compound of Formula (I), or a pharmaceutically acceptable salt thereof, may decrease the level of NO in cultured cells treated with palmitate, as illustrated in FIG. 5H for administering Compound A. The blood alanine transaminase (ALT) level is increased in mice fed on HFD, as illustrated in FIG. 5I. In some embodiments, administering a compound of Formula (I), or a pharmaceutically acceptable salt thereof, may decrease the level of ALT, as illustrated in FIG. 5I for administering Compound A. In some embodiments, the compound of Formula (I), or a pharmaceutically acceptable salt thereof, is Compound A.

Another embodiment provides a method for treating obesity comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Another embodiment provides a method for reducing mitochondrial stress in a subject, comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Another embodiment provides a method for increasing fatty acid oxidation (FAO) activity in a subject comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Another embodiment provides a method for treating a disease or disorder (such as inflammation, diabetes, nonalcoholic fatty liver disease (NAFLD) or nonalcoholic steatohepatitis (NASH), etc.), in a subject in need thereof, comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Another embodiment provides a compound of Formula (I), or a pharmaceutically acceptable salt thereof, for use in a method of treatment of a disease or disorder (such as inflammation, diabetes, nonalcoholic fatty liver disease (NAFLD) or nonalcoholic steatohepatitis (NASH), etc.), in a subject in need thereof, comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Another embodiment provides a pharmaceutical composition comprising a compound of Formula (I), or a pharmaceutically acceptable salt thereof, and a pharmaceutically acceptable excipient for use in a method for treatment of a disease or disorder (such as inflammation, diabetes, nonalcoholic fatty liver disease (NAFLD) or nonalcoholic steatohepatitis (NASH), etc.), in a subject in need thereof, comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Another embodiment provides a use of a compound of Formula (I), or a pharmaceutically acceptable salt thereof, in the manufacture of a medicament for treatment of a disease or disorder (such as inflammation, diabetes, nonalcoholic fatty liver disease (NAFLD) or nonalcoholic steatohepatitis (NASH), etc.), in a subject in need thereof, comprising administering to the subject a therapeutically effective amount of a compound of Formula (I), or a pharmaceutically acceptable salt thereof. In some embodiments, the compound of Formula (I), or a pharmaceutically acceptable salt thereof, is Compound A, or a pharmaceutically acceptable salt thereof.

Doses employed for adult human treatment are typically in the range of 0.1 mg-40,000 mg per day. Oral doses typically range from about 1.0 mg to about 40,000 mg, one to four times, or more, per day.

In some embodiments, the dose of a compound of Formula (I), or a pharmaceutically acceptable salt thereof, is about 0.1 mg per day to about 40,000 mg per day. In some embodiments, the dose of a compound of Formula (I), or a pharmaceutically acceptable salt thereof, is about 0.1 mg per day to about 1 mg per day, about 0.1 mg per day to about 10 mg per day, about 0.1 mg per day to about 50 mg per day, about 0.1 mg per day to about 100 mg per day, about 0.1 mg per day to about 300 mg per day, about 0.1 mg per day to about 500 mg per day, about 0.1 mg per day to about 600 mg per day, about 0.1 mg per day to about 700 mg per day, about 0.1 mg per day to about 800 mg per day, about 0.1 mg per day to about 900 mg per day, about 0.1 mg per day to about 1,000 mg per day, about 0.1 mg per day to about 5,000 mg per day, about 0.1 mg per day to about 10,000 mg per day, about 0.1 mg per day to about 20,000 mg per day, about 0.1 mg per day to about 30,000 mg per day, about 0.1 mg per day to about 40,000 mg per day, about 1 mg per day to about 10 mg per day, about 1 mg per day to about 50 mg per day, about 1 mg per day to about 100 mg per day, about 1 mg per day to about 300 mg per day, about 1 mg per day to about 500 mg per day, about 1 mg per day to about 600 mg per day, about 1 mg per day to about 700 mg per day, about 1 mg per day to about 800 mg per day, about 1 mg per day to about 900 mg per day, about 1 mg per day to about 1,000 mg per day, about 1 mg per day to about 5,000 mg per day, about 1 mg per day to about 10,000 mg per day, about 1 mg per day to about 20,000 mg per day, about 1 mg per day to about 30,000 mg per day, about 1 mg per day to about 40,000 mg per day, about 10 mg per day to about 50 mg per day, about 10 mg per day to about 100 mg per day, about 10 mg per day to about 300 mg per day, about 10 mg per day to about 500 mg per day, about 10 mg per day to about 600 mg per day, about 10 mg per day to about 700 mg per day, about 10 mg per day to about 800 mg per day, about 10 mg per day to about 900 mg per day, about 10 mg per day to about 1,000 mg per day, about 10 mg per day to about 5,000 mg per day, about 10 mg per day to about 10,000 mg per day, about 10 mg per day to about 20,000 mg per day, about 10 mg per day to about 30,000 mg per day, about 10 mg per day to about 40,000 mg per day, about 50 mg per day to about 100 mg per day, about 50 mg per day to about 300 mg per day, about 50 mg per day to about 500 mg per day, about 50 mg per day to about 600 mg per day, about 50 mg per day to about 700 mg per day, about 50 mg per day to about 800 mg per day, about 50 mg per day to about 900 mg per day, about 50 mg per day to about 1,000 mg per day, about 50 mg per day to about 5,000 mg per day, about 50 mg per day to about 10,000 mg per day, about 50 mg per day to about 20,000 mg per day, about 50 mg per day to about 30,000 mg per day, about 50 mg per day to about 40,000 mg per day, about 100 mg per day to about 300 mg per day, about 100 mg per day to about 500 mg per day, about 100 mg per day to about 600 mg per day, about 100 mg per day to about 700 mg per day, about 100 mg per day to about 800 mg per day, about 100 mg per day to about 900 mg per day, about 100 mg per day to about 1,000 mg per day, about 100 mg per day to about 5,000 mg per day, about 100 mg per day to about 10,000 mg per day, about 100 mg per day to about 20,000 mg per day, about 100 mg per day to about 30,000 mg per day, about 100 mg per day to about 40,000 mg per day, about 300 mg per day to about 500 mg per day, about 300 mg per day to about 600 mg per day, about 300 mg per day to about 700 mg per day, about 300 mg per day to about 800 mg per day, about 300 mg per day to about 900 mg per day, about 300 mg per day to about 1,000 mg per day, about 300 mg per day to about 5,000 mg per day, about 300 mg per day to about 10,000 mg per day, about 300 mg per day to about 20,000 mg per day, about 300 mg per day to about 30,000 mg per day, about 300 mg per day to about 40,000 mg per day, about 500 mg per day to about 600 mg per day, about 500 mg per day to about 700 mg per day, about 500 mg per day to about 800 mg per day, about 500 mg per day to about 900 mg per day, about 500 mg per day to about 1,000 mg per day, about 500 mg per day to about 5,000 mg per day, about 500 mg per day to about 10,000 mg per day, about 500 mg per day to about 20,000 mg per day, about 500 mg per day to about 30,000 mg per day, about 500 mg per day to about 40,000 mg per day, about 600 mg per day to about 700 mg per day, about 600 mg per day to about 800 mg per day, about 600 mg per day to about 900 mg per day, about 600 mg per day to about 1,000 mg per day, about 600 mg per day to about 5,000 mg per day, about 600 mg per day to about 10,000 mg per day, about 600 mg per day to about 20,000 mg per day, about 600 mg per day to about 30,000 mg per day, about 600 mg per day to about 40,000 mg per day, about 700 mg per day to about 800 mg per day, about 700 mg per day to about 900 mg per day, about 700 mg per day to about 1,000 mg per day, about 700 mg per day to about 5,000 mg per day, about 700 mg per day to about 10,000 mg per day, about 700 mg per day to about 20,000 mg per day, about 700 mg per day to about 30,000 mg per day, about 700 mg per day to about 40,000 mg per day, about 800 mg per day to about 900 mg per day, about 800 mg per day to about 1,000 mg per day, about 800 mg per day to about 5,000 mg per day, about 800 mg per day to about 10,000 mg per day, about 800 mg per day to about 20,000 mg per day, about 800 mg per day to about 30,000 mg per day, about 800 mg per day to about 40,000 mg per day, about 900 mg per day to about 1,000 mg per day, about 900 mg per day to about 5,000 mg per day, about 900 mg per day to about 10,000 mg per day, about 900 mg per day to about 20,000 mg per day, about 900 mg per day to about 30,000 mg per day, about 900 mg per day to about 40,000 mg per day, about 1,000 mg per day to about 5,000 mg per day, about 1,000 mg per day to about 10,000 mg per day about 1,000 mg per day to about 20,000 mg per day about 1,000 mg per day to about 30,000 mg per day about 1,000 mg per day to about 40,000 mg per day, about 5,000 mg per day to about 10,000 mg per day, about 5,000 mg per day to about 20,000 mg per day, about 5,000 mg per day to about 30,000 mg per day, about 5,000 mg per day to about 40,000 mg per day, about 10,000 mg per day to about 20,000 mg per day, about 10,000 mg per day to about 30,000 mg per day, about 10,000 mg per day to about 40,000 mg per day, about 20,000 mg per day to about 30,000 mg per day, about 20,000 mg per day to about 40,000 mg per day, or about 30,000 mg per day to about 40,000 mg per day. In some embodiments, the dose of a compound of Formula (I), or a pharmaceutically acceptable salt thereof, is about 0.1 mg per day, about 1 mg per day, about 10 mg per day, about 50 mg per day, about 100 mg per day, about 300 mg per day, about 500 mg per day, about 600 mg per day, about 700 mg per day, about 800 mg per day, about 900 mg per day, about 1,000 mg per day, about 5,000 mg per day, about 10,000 mg per day, about 20,000 mg per day, about 30,000 mg per day, or about 40,000 mg per day. In some embodiments, the dose of a compound of Formula (I), or a pharmaceutically acceptable salt thereof, is at least about 0.1 mg per day, at least about 1 mg per day, at least about 10 mg per day, at least about 50 mg per day, at least about 100 mg per day, at least about 300 mg per day, at least about 500 mg per day, at least about 600 mg per day, at least about 700 mg per day, at least about 800 mg per day, at least about 900 mg per day, at least about 1000 mg per day, at least about 5,000 mg per day, at least about 10,000 mg per day, at least about 20,000 mg per day, at least about 30,000 mg per day, or at least about 40,000 mg per day. In some embodiments, the dose of a compound of Formula (I), or a pharmaceutically acceptable salt thereof, is at most about 1 mg per day, at most about 10 mg per day, at most about 50 mg per day, at most about 100 mg per day, at most about 300 mg per day, at most about 500 mg per day, at most about 600 mg per day, at most about 700 mg per day, at most about 800 mg per day, at most about 900 mg per day, at most about 1,000 mg per day, at most about 5,000 mg per day, at most about 10,000 mg per day, at most about 20,000 mg per day, at most about 30,000 mg per day, or at most about 40,000 mg per day. In some embodiments, the compound of Formula (I), or a pharmaceutically acceptable salt thereof, is Compound A, or a pharmaceutically acceptable salt thereof.

In some embodiments, the oral dose of a compound of Formula (I), or a pharmaceutically acceptable salt thereof, is about 1 mg per day to about 40,000 mg per day. In some embodiments, the oral dose of a compound of Formula (I), or a pharmaceutically acceptable salt thereof, is about 1 mg per day to about 10 mg per day, about 1 mg per day to about 20 mg per day, about 1 mg per day to about 50 mg per day, about 1 mg per day to about 100 mg per day, about 1 mg per day to about 200 mg per day, about 1 mg per day to about 300 mg per day, about 1 mg per day to about 400 mg per day, about 1 mg per day to about 500 mg per day, about 1 mg per day to about 600 mg per day, about 1 mg per day to about 700 mg per day, about 1 mg per day to about 800 mg per day, about 1 mg per day to about 900 mg per day, about 1 mg per day to about 1,000 mg per day, about 1 mg per day to about 2,000 mg per day, about 1 mg per day to about 3,000 mg per day, about 1 mg per day to about 5,000 mg per day, about 1 mg per day to about 10,000 mg per day, about 1 mg per day to about 20,000 mg per day, about 1 mg per day to about 30,000 mg per day, about 1 mg per day to about 40,000 mg per day, about 10 mg per day to about 20 mg per day, about 10 mg per day to about 50 mg per day, about 10 mg per day to about 100 mg per day, about 10 mg per day to about 200 mg per day, about 10 mg per day to about 300 mg per day, about 10 mg per day to about 400 mg per day, about 10 mg per day to about 500 mg per day, about 10 mg per day to about 600 mg per day, about 10 mg per day to about 700 mg per day, about 10 mg per day to about 800 mg per day, about 10 mg per day to about 900 mg per day, about 10 mg per day to about 1,000 mg per day, about 10 mg per day to about 2,000 mg per day, about 10 mg per day to about 3,000 mg per day, about 10 mg per day to about 5,000 mg per day, about 10 mg per day to about 10,000 mg per day, about 10 mg per day to about 20,000 mg per day, about 10 mg per day to about 30,000 mg per day, about 10 mg per day to about 40,000 mg per day, about 20 mg per day to about 50 mg per day, about 20 mg per day to about 100 mg per day, about 20 mg per day to about 200 mg per day, about 20 mg per day to about 300 mg per day, about 20 mg per day to about 400 mg per day, about 20 mg per day to about 500 mg per day, about 20 mg per day to about 600 mg per day, about 20 mg per day to about 700 mg per day, about 20 mg per day to about 800 mg per day, about 20 mg per day to about 900 mg per day, about 20 mg per day to about 1,000 mg per day, about 20 mg per day to about 2,000 mg per day, about 20 mg per day to about 3,000 mg per day, about 20 mg per day to about 5,000 mg per day, about 20 mg per day to about 10,000 mg per day, about 20 mg per day to about 20,000 mg per day, about 20 mg per day to about 30,000 mg per day, about 20 mg per day to about 40,000 mg per day, about 50 mg per day to about 100 mg per day, about 50 mg per day to about 200 mg per day, about 50 mg per day to about 300 mg per day, about 50 mg per day to about 400 mg per day, about 50 mg per day to about 500 mg per day, about 50 mg per day to about 600 mg per day, about 50 mg per day to about 700 mg per day, about 50 mg per day to about 800 mg per day, about 50 mg per day to about 900 mg per day, about 50 mg per day to about 1,000 mg per day, about 50 mg per day to about 2,000 mg per day, about 50 mg per day to about 3,000 mg per day, about 50 mg per day to about 5,000 mg per day, about 50 mg per day to about 10,000 mg per day, about 50 mg per day to about 20,000 mg per day, about 50 mg per day to about 30,000 mg per day, about 50 mg per day to about 40,000 mg per day, about 100 mg per day to about 200 mg per day, about 100 mg per day to about 300 mg per day, about 100 mg per day to about 400 mg per day, about 100 mg per day to about 500 mg per day, about 100 mg per day to about 600 mg per day, about 100 mg per day to about 700 mg per day, about 100 mg per day to about 800 mg per day, about 100 mg per day to about 900 mg per day, about 100 mg per day to about 1,000 mg per day, about 100 mg per day to about 2,000 mg per day, about 100 mg per day to about 3,000 mg per day, about 100 mg per day to about 5,000 mg per day, about 100 mg per day to about 10,000 mg per day, about 100 mg per day to about 20,000 mg per day, about 100 mg per day to about 30,000 mg per day, about 100 mg per day to about 40,000 mg per day, about 200 mg per day to about 300 mg per day, about 200 mg per day to about 400 mg per day, about 200 mg per day to about 500 mg per day, about 200 mg per day to about 600 mg per day, about 200 mg per day to about 700 mg per day, about 200 mg per day to about 800 mg per day, about 200 mg per day to about 900 mg per day, about 200 mg per day to about 1,000 mg per day, about 200 mg per day to about 2,000 mg per day, about 200 mg per day to about 3,000 mg per day, about 200 mg per day to about 5,000 mg per day, about 200 mg per day to about 10,000 mg per day, about 200 mg per day to about 20,000 mg per day, about 200 mg per day to about 30,000 mg per day, about 200 mg per day to about 40,000 mg per day, about 300 mg per day to about 400 mg per day, about 300 mg per day to about 500 mg per day, about 300 mg per day to about 600 mg per day, about 300 mg per day to about 700 mg per day, about 300 mg per day to about 800 mg per day, about 300 mg per day to about 900 mg per day, about 300 mg per day to about 1,000 mg per day, about 300 mg per day to about 2,000 mg per day, about 300 mg per day to about 3,000 mg per day, about 300 mg per day to about 5,000 mg per day, about 300 mg per day to about 10,000 mg per day, about 300 mg per day to about 20,000 mg per day, about 300 mg per day to about 30,000 mg per day, about 300 mg per day to about 40,000 mg per day, about 400 mg per day to about 500 mg per day, about 400 mg per day to about 600 mg per day, about 400 mg per day to about 700 mg per day, about 400 mg per day to about 800 mg per day, about 400 mg per day to about 900 mg per day, about 400 mg per day to about 1,000 mg per day, about 400 mg per day to about 2,000 mg per day, about 400 mg per day to about 3,000 mg per day, about 400 mg per day to about 5,000 mg per day, about 400 mg per day to about 10,000 mg per day, about 400 mg per day to about 20,000 mg per day, about 400 mg per day to about 30,000 mg per day, about 400 mg per day to about 40,000 mg per day, about 500 mg per day to about 600 mg per day, about 500 mg per day to about 700 mg per day, about 500 mg per day to about 800 mg per day, about 500 mg per day to about 900 mg per day, about 500 mg per day to about 1,000 mg per day, about 500 mg per day to about 2,000 mg per day, about 500 mg per day to about 3,000 mg per day, about 500 mg per day to about 5,000 mg per day, about 500 mg per day to about 10,000 mg per day, about 500 mg per day to about 20,000 mg per day, about 500 mg per day to about 30,000 mg per day, about 500 mg per day to about 40,000 mg per day, about 600 mg per day to about 700 mg per day, about 600 mg per day to about 800 mg per day, about 600 mg per day to about 900 mg per day, about 600 mg per day to about 1,000 mg per day, about 600 mg per day to about 2,000 mg per day, about 600 mg per day to about 3,000 mg per day, about 600 mg per day to about 5,000 mg per day, about 600 mg per day to about 10,000 mg per day, about 600 mg per day to about 20,000 mg per day, about 600 mg per day to about 30,000 mg per day, about 600 mg per day to about 40,000 mg per day, about 700 mg per day to about 800 mg per day, about 700 mg per day to about 900 mg per day, about 700 mg per day to about 1,000 mg per day, about 700 mg per day to about 2,000 mg per day, about 700 mg per day to about 3,000 mg per day, about 700 mg per day to about 5,000 mg per day, about 700 mg per day to about 10,000 mg per day, about 700 mg per day to about 20,000 mg per day, about 700 mg per day to about 30,000 mg per day, about 700 mg per day to about 40,000 mg per day, about 800 mg per day to about 900 mg per day, about 800 mg per day to about 1,000 mg per day, about 800 mg per day to about 2,000 mg per day, about 800 mg per day to about 3,000 mg per day, about 800 mg per day to about 5,000 mg per day, about 800 mg per day to about 10,000 mg per day, about 800 mg per day to about 20,000 mg per day, about 800 mg per day to about 30,000 mg per day, about 800 mg per day to about 40,000 mg per day, about 900 mg per day to about 1,000 mg per day, about 900 mg per day to about 2,000 mg per day, about 900 mg per day to about 3,000 mg per day, about 900 mg per day to about 5,000 mg per day, about 900 mg per day to about 10,000 mg per day, about 900 mg per day to about 20,000 mg per day, about 900 mg per day to about 30,000 mg per day, about 900 mg per day to about 40,000 mg per day, about 1,000 mg per day to about 2,000 mg per day, about 1,000 mg per day to about 3,000 mg per day, about 1,000 mg per day to about 5,000 mg per day, about 1,000 mg per day to about 10,000 mg per day, about 1,000 mg per day to about 20,000 mg per day, about 1,000 mg per day to about 30,000 mg per day, about 1,000 mg per day to about 40,000 mg per day, about 2,000 mg per day to about 3,000 mg per day, about 2,000 mg per day to about 5,000 mg per day, about 2,000 mg per day to about 10,000 mg per day, about 2,000 mg per day to about 20,000 mg per day, about 2,000 mg per day to about 30,000 mg per day, about 2,000 mg per day to about 40,000 mg per day, about 3,000 mg per day to about 5,000 mg per day, about 3,000 mg per day to about 10,000 mg per day, about 3,000 mg per day to about 20,000 mg per day, about 3,000 mg per day to about 30,000 mg per day, about 3,000 mg per day to about 40,000 mg per day, about 5,000 mg per day to about 10,000 mg per day, about 5,000 mg per day to about 20,000 mg per day, about 5,000 mg per day to about 30,000 mg per day, about 5,000 mg per day to about 40,000 mg per day, about 10,000 mg per day to about 20,000 mg per day, about 10,000 mg per day to about 30,000 mg per day, about 10,000 mg per day to about 40,000 mg per day, about 20,000 mg per day to about 30,000 mg per day, about 20,000 mg per day to about 40,000 mg per day, or about 30,000 mg per day to about 40,000 mg per day. In some embodiments, the oral dose of a compound of Formula (I), or a pharmaceutically acceptable salt thereof, is about 1 mg per day, about 10 mg per day, about 20 mg per day, about 50 mg per day, about 100 mg per day, about 200 mg per day, about 300 mg per day, about 400 mg per day, about 500 mg per day, about 600 mg per day, about 700 mg per day, about 800 mg per day, about 900 mg per day, about 1,000 mg per day, about 2,000 mg per day, about 3,000 mg per day, about 5,000 mg per day, about 10,000 mg per day, about 20,000 mg per day, about 30,000 mg per day, or about 40,000 mg per day. In some embodiments, the oral dose of a compound of Formula (I), or a pharmaceutically acceptable salt thereof, is at least about 1 mg per day, at least about 10 mg per day, at least about 20 mg per day, at least about 50 mg per day, at least about 100 mg per day, at least about 200 mg per day, at least about 300 mg per day, at least about 400 mg per day, at least about 500 mg per day, at least about 600 mg per day, at least about 700 mg per day, at least about 800 mg per day, at least about 900 mg per day, at least about 1,000 mg per day, at least about 2,000 mg per day, at least about 3,000 mg per day, at least about 5,000 mg per day, at least about 10,000 mg per day, at least about 20,000 mg per day, at least about 30,000 mg per day, or at least about 40,000 mg per day. In some embodiments, the oral dose of a compound of Formula (I), or a pharmaceutically acceptable salt thereof, is at most about 10 mg per day, at most about 20 mg per day, at most about 50 mg per day, at most about 100 mg per day, at most about 200 mg per day, at most about 300 mg per day, at most about 400 mg per day, at most about 500 mg per day, at most about 600 mg per day, at most about 700 mg per day, at most about 800 mg per day, at most about 900 mg per day, at most about 1,000 mg per day, at most about 2,000 mg per day, at most about 3,000 mg per day, at most about 5,000 mg per day, at most about 10,000 mg per day, at most about 20,000 mg per day, at most about 30,000 mg per day, or at most about 40,000 mg per day. In some embodiments, the compound of Formula (I), or a pharmaceutically acceptable salt thereof, is Compound A, or a pharmaceutically acceptable salt thereof.

In some embodiments, the dose of a compound of Formula (I), or a pharmaceutically acceptable salt thereof, is about 10 mg per kg per day to about 500 mg per kg per day. In some embodiments, the dose of a compound of Formula (I), or a pharmaceutically acceptable salt thereof, is about 10 mg per kg per day to about 15 mg per kg per day, about 10 mg per kg per day to about 20 mg per kg per day, about 10 mg per kg per day to about 25 mg per kg per day, about 10 mg per kg per day to about 30 mg per kg per day, about 10 mg per kg per day to about 40 mg per kg per day, about 10 mg per kg per day to about 50 mg per kg per day, about 10 mg per kg per day to about 100 mg per kg per day, about 10 mg per kg per day to about 200 mg per kg per day, about 10 mg per kg per day to about 300 mg per kg per day, about 10 mg per kg per day to about 400 mg per kg per day, about 10 mg per kg per day to about 500 mg per kg per day, about 15 mg per kg per day to about 20 mg per kg per day, about 15 mg per kg per day to about 25 mg per kg per day, about 15 mg per kg per day to about 30 mg per kg per day, about 15 mg per kg per day to about 40 mg per kg per day, about 15 mg per kg per day to about 50 mg per kg per day, about 15 mg per kg per day to about 100 mg per kg per day, about 15 mg per kg per day to about 200 mg per kg per day, about 15 mg per kg per day to about 300 mg per kg per day, about 15 mg per kg per day to about 400 mg per kg per day, about 15 mg per kg per day to about 500 mg per kg per day, about 20 mg per kg per day to about 25 mg per kg per day, about 20 mg per kg per day to about 30 mg per kg per day, about 20 mg per kg per day to about 40 mg per kg per day, about 20 mg per kg per day to about 50 mg per kg per day, about 20 mg per kg per day to about 100 mg per kg per day, about 20 mg per kg per day to about 200 mg per kg per day, about 20 mg per kg per day to about 300 mg per kg per day, about 20 mg per kg per day to about 400 mg per kg per day, about 20 mg per kg per day to about 500 mg per kg per day, about 25 mg per kg per day to about 30 mg per kg per day, about 25 mg per kg per day to about 40 mg per kg per day, about 25 mg per kg per day to about 50 mg per kg per day, about 25 mg per kg per day to about 100 mg per kg per day, about 25 mg per kg per day to about 200 mg per kg per day, about 25 mg per kg per day to about 300 mg per kg per day, about 25 mg per kg per day to about 400 mg per kg per day, about 25 mg per kg per day to about 500 mg per kg per day, about 30 mg per kg per day to about 40 mg per kg per day, about 30 mg per kg per day to about 50 mg per kg per day, about 30 mg per kg per day to about 100 mg per kg per day, about 30 mg per kg per day to about 200 mg per kg per day, about 30 mg per kg per day to about 300 mg per kg per day, about 30 mg per kg per day to about 400 mg per kg per day, about 30 mg per kg per day to about 500 mg per kg per day, about 40 mg per kg per day to about 50 mg per kg per day, about 40 mg per kg per day to about 100 mg per kg per day, about 40 mg per kg per day to about 200 mg per kg per day, about 40 mg per kg per day to about 300 mg per kg per day, about 40 mg per kg per day to about 400 mg per kg per day, about 40 mg per kg per day to about 500 mg per kg per day, about 50 mg per kg per day to about 100 mg per kg per day, about 50 mg per kg per day to about 200 mg per kg per day, about 50 mg per kg per day to about 300 mg per kg per day, about 50 mg per kg per day to about 400 mg per kg per day, about 50 mg per kg per day to about 500 mg per kg per day, about 100 mg per kg per day to about 200 mg per kg per day, about 100 mg per kg per day to about 300 mg per kg per day, about 100 mg per kg per day to about 400 mg per kg per day, about 100 mg per kg per day to about 500 mg per kg per day, about 200 mg per kg per day to about 300 mg per kg per day, about 200 mg per kg per day to about 400 mg per kg per day, about 200 mg per kg per day to about 500 mg per kg per day, about 300 mg per kg per day to about 400 mg per kg per day, about 300 mg per kg per day to about 500 mg per kg per day, or about 400 mg per kg per day to about 500 mg per kg per day. In some embodiments, the dose of a compound of Formula (I), or a pharmaceutically acceptable salt thereof, is about 10 mg per kg per day, about 15 mg per kg per day, about 20 mg per kg per day, about 25 mg per kg per day, about 30 mg per kg per day, about 40 mg per kg per day, about 50 mg per kg per day, about 100 mg per kg per day, about 200 mg per kg per day, about 300 mg per kg per day, about 400 mg per kg per day, or about 500 mg per kg per day. In some embodiments, the dose of a compound of Formula (I), or a pharmaceutically acceptable salt thereof, is at least about 10 mg per kg per day, at least about 15 mg per kg per day, at least about 20 mg per kg per day, at least about 25 mg per kg per day, at least about 30 mg per kg per day, at least about 40 mg per kg per day, at least about 50 mg per kg per day, at least about 100 mg per kg per day, at least about 200 mg per kg per day, at least about 300 mg per kg per day, at least about 400 mg per kg per day, or at least about 500 mg per kg per day. In some embodiments, the dose of a compound of Formula (I), or a pharmaceutically acceptable salt thereof, is at most about 10 mg per kg per day, at most about 15 mg per kg per day, at most about 20 mg per kg per day, at most about 25 mg per kg per day, at most about 30 mg per kg per day, at most about 40 mg per kg per day, at most about 50 mg per kg per day, at most about 100 mg per kg per day, at most about 200 mg per kg per day, at most about 300 mg per kg per day, at most about 400 mg per kg per day, or at most about 500 mg per kg per day. In some embodiments, the compound of Formula (I), or a pharmaceutically acceptable salt thereof, is Compound A, or a pharmaceutically acceptable salt thereof.

In some embodiments, the dosage of a compound of Formula (I), or a pharmaceutically acceptable salt thereof, is administered once every six hours, once every eight hours, once every twelve hours, or once every twenty-four hours. In some embodiments, the compound of Formula (I), or a pharmaceutically acceptable salt thereof, is Compound A, or a pharmaceutically acceptable salt thereof.

In some embodiments, the dosage of a compound of Formula (I), or a pharmaceutically acceptable salt thereof, is administered once a day, once every two days, once every three days, once every week, once every two weeks, or once every month. In some embodiments, the compound of Formula (I), or a pharmaceutically acceptable salt thereof, is Compound A, or a pharmaceutically acceptable salt thereof.

In some embodiments, the dosage of a compound of Formula (I), or a pharmaceutically acceptable salt thereof, is administered once a day (q.d.), twice a day (b.i.d.), three times a day (t.i.d.), or four times a day (q.i.d.). In some embodiments, the compound of Formula (I), or a pharmaceutically acceptable salt thereof, is Compound A, or a pharmaceutically acceptable salt thereof.

In some embodiments, the compound of Formula (I), or a pharmaceutically acceptable salt thereof, is administered for about 1 day, about 2 days, about 5 days, about 7 days, about 10 days, about 15 days, about 30 days, about 60 days, about 90 days, about 120 days, about 180 days, about 365 days, or about 730 days. In some embodiments, the compound of Formula (I), or a pharmaceutically acceptable salt thereof, is administered for at least about 1 days, at least about 2 days, at least about 5 days, at least about 7 days, at least about 10 days, at least about 15 days, at least about 30 days, at least about 60 days, at least about 90 days, at least about 120 days, at least about 180 days, at least about 365 days, or at least 730 days. In some embodiments, the compound of Formula (I), or a pharmaceutically acceptable salt thereof, is administered for about 1 week, about 2 weeks, about 3 weeks, about 4 weeks, about 6 weeks, about 8 weeks, about 10 weeks, about 12 weeks, about 16 weeks, about 20 weeks, about 26 weeks, about 39 weeks, about 52 weeks, about 78 weeks, or about 104 weeks. In some embodiments, the compound of Formula (I), or a pharmaceutically acceptable salt thereof, is administered for at least about 1 week, at least about 2 weeks, at least about 3 weeks, at least about 4 weeks, at least about 6 weeks, at least about 8 weeks, at least about 10 weeks, at least about 12 weeks, at least about 16 weeks, at least about 20 weeks, at least about 26 weeks, at least about 39 weeks, at least about 52 weeks, at least about 78 weeks, or at least about 104 weeks. In some embodiments, a compound of Formula (I), or a pharmaceutically acceptable salt thereof, is administered for about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months. In some embodiments, a compound of Formula (I), or a pharmaceutically acceptable salt thereof, is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 15 months, at least about 18 months, or at least about 24 months. In some embodiments, the compound of Formula (I), or a pharmaceutically acceptable salt thereof, is Compound A, or a pharmaceutically acceptable salt thereof.

Non-alcoholic fatty liver disease (NAFLD), also known as metabolic (dysfunction) associated fatty liver disease (MAFLD), is excessive fat build-up in the liver without another clear cause such as alcohol use. There are two types of NAFLD, non-alcoholic fatty liver (NAFL) and non-alcoholic steatohepatitis (NASH), with the latter also including liver inflammation. NAFL is less dangerous than NASH and usually does not progress to NASH or liver cirrhosis. When NAFL does progress to NASH, it may eventually lead to complications such as cirrhosis, liver cancer, liver failure, or cardiovascular disease. The term NAFLD encompasses a continuum of liver abnormalities, from non-alcoholic fatty liver (NAFL, simple steatosis) to non-alcoholic steatohepatitis (NASH). A liver can remain fatty without disturbing liver function (NAFL), but by various mechanisms and possible insults to the liver, it may also progress into non-alcoholic steatohepatitis (NASH), a state in which steatosis is combined with inflammation and sometimes fibrosis (steatohepatitis). NASH can then lead to complications such as cirrhosis and hepatocellular carcinoma.

NAFLD is the most common liver disorder worldwide and is present in approximately 25% of the world's population. It is very common in developed nations, such as the United States, and affected about 75 to 100 million Americans in 2017. Over 90% of obese, 60% of diabetic, and up to 20% of normal-weight people develop it. NAFLD is the leading cause of chronic liver disease and the second most common reason for liver transplantation in the US and Europe as of 2017. NAFLD affects about 20 to 25% of people in Europe. In the United States, estimates suggest between 30 and 40% of adults have NAFLD, and about 3 to 12% of adults have NASH. The annual economic burden was approximately US$103 billion in the US in 2016.

One embodiment provides a compound of Formula (I), or a pharmaceutically acceptable salt thereof, for use in a method for treatment of a disease or disorder (such as inflammation, diabetes, nonalcoholic fatty liver disease (NAFLD) or nonalcoholic steatohepatitis (NASH), etc.), in a subject in need thereof. In some embodiments, the compound of Formula (I), or a pharmaceutically acceptable salt thereof, is Compound A, or a pharmaceutically acceptable salt thereof.

One embodiment provides a pharmaceutical composition comprising a compound of Formula (I), or a pharmaceutically acceptable salt or solvate thereof, and a pharmaceutically acceptable excipient for use in a method for treatment of a disease or disorder (such as inflammation, diabetes, nonalcoholic fatty liver disease (NAFLD) or nonalcoholic steatohepatitis (NASH), etc.), in a subject in need thereof. In some embodiments, the compound of Formula (I), or a pharmaceutically acceptable salt thereof, is Compound A, or a pharmaceutically acceptable salt thereof.

One embodiment provides a use of a compound of Formula (I), or a pharmaceutically acceptable salt thereof, in the manufacture of a medicament for the treatment of a disease or disorder (such as inflammation, diabetes, nonalcoholic fatty liver disease (NAFLD) or nonalcoholic steatohepatitis (NASH), etc.), in a subject in need thereof. In some embodiments, the compound of Formula (I), or a pharmaceutically acceptable salt thereof, is Compound A, or a pharmaceutically acceptable salt thereof.

In some embodiments, the disease or disorder is an inflammatory and autoimmune disease or disorder. In some embodiments, the disease or disorder is inflammation. In some embodiments, the inflammatory and autoimmune disease or disorder is selected from, but are not limited to: rheumatoid arthritis, psoriatic arthritis, systemic onset juvenile idiopathic arthritis, multiple sclerosis, lupus nephritis, systemic lupus erythematosus, psoriasis, Crohn's disease, colitis, asthma, graft versus host disease, allograft rejection, chronic obstructive pulmonary disease, multiple sclerosis, Alzheimer's disease, Graves' disease, cutaneous lupus, ankylosing spondylitis, cryopyrin-associated periodic syndromes (CAPS), gout, and gouty arthritis, ulcerative TNF receptor associated periodic syndrome (TRAPS), Wegener's granulomatosis, sarcoidosis, familial Mediterranean fever (FMF), neuropathic pain, and adult onset stills.

Overweight and obesity increase the risk for many health problems, such as type 2 diabetes, high blood pressure, heart disease, stroke, joint problems, liver disease, gallstones, some types of cancer, and sleep and breathing problems, kidney disease, as well as gestational diabetes, preeclampsia (high blood pressure) during pregnancy.

Strong risk factors for NAFLD include obesity and type 2 diabetes. Other risks include being overweight, metabolic syndrome (defined as at least three of the five of the following medical conditions: abdominal obesity, high blood pressure, high blood sugar, high serum triglycerides, and low serum HDL cholesterol), a diet high in fructose, and older age. NAFLD and alcoholic liver disease are types of fatty liver disease.

Diabetes, also known as diabetes mellitus, is a group of metabolic disorders characterized by a high blood sugar level (hyperglycemia) over a prolonged period of time. Diabetes is due to either the pancreas not producing enough insulin, or the cells of the body not responding properly to the insulin produced. Insulin is a hormone which is responsible for helping glucose from food get into cells to be used for energy.

Type 2 diabetes begins with insulin resistance, a condition in which cells fail to respond to insulin properly. As the disease progresses, a lack of insulin may also develop. Type 2 diabetes makes up about 90% of cases of diabetes. Type 2 diabetes is more common in older adults, but a significant increase in the prevalence of obesity among children has led to more cases of type 2 diabetes in younger people. The most common cause is a combination of excessive body weight and insufficient exercise. Other critical factors for type 2 diabetes include aging and lipotoxic effects on pancreatic B-cells over time. The liver plays a key role in type 2 diabetes, where the predominant paradigm is that cellular stress and inflammation contribute to insulin resistance and dysregulated hepatic gluconeogenesis.

Provided herein is the method wherein the pharmaceutical composition is administered orally. Provided herein is the method wherein the pharmaceutical composition is administered by injection. Depending on the disorder, disease, or condition to be treated, and the subject's condition, the compounds or pharmaceutical compositions provided herein can be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical (e.g., transdermal or local) routes of administration and can be formulated, alone or together, in suitable dosage unit with pharmaceutically acceptable excipients, carriers, adjuvants, and vehicles appropriate for each route of administration as described elsewhere herein.

Other embodiments and uses will be apparent to one skilled in the art in light of the present disclosures. The following examples are provided merely as illustrative of various embodiments and shall not be construed to limit the invention in any way.

### Examples

### Example 1 In Vivo Mouse Experiments

NCT oral administration. Four-week-old male C57BL/6J (JAX cat #000664) mice were purchased from Jackson laboratory and were maintained in a 12 h light/dark cycle throughout the experiment. Prior to the experiments, mice were acclimated for 2 weeks. Six-week-old mice with similar body weights were randomized to a normal diet (NC), a high fat diet (HFD) (Research Diets, cat #D12492 60 kcal % fat) or HFD containing 4000 ppm NCT (HFD+NCT) (Research Diets, 60 kcal % fat+4000 ppm NCT), which was calculated to provide approximately 400 mg/kg/d NCT. HFD chow containing NCT was made with gray dye to distinguish it from regular HFD chow that had green dye. Mice for each treatment group were placed in separate cages. Equal amounts of fresh chow were provided every week to all mice. Body weight gain and food intake were measured every week for 10 weeks. After 10 weeks of chow treatment, mice were sacrificed for analysis. All animal experiments were approved by the Institutional Animal Care and Use Committee (IACUC) of the Sanford Burnham Prebys Medical Discovery Institute in accordance with national regulations. Sample size was chosen based on the prior study of NCT delivered by IP injection.

Subcutaneous and oral gavage treatment. Twelve-week-old C57BL/6J DIO male mice were purchased from Jackson laboratory (cat #380050) and were fed with HFD (Research Diets, cat #D12492 60 kcal % fat). Prior to the experiments, mice were acclimated for 2 weeks. Fourteen-week-old mice were injected subcutaneously using sterile insulin syringes filled with DMSO or NCT (200 mg/kg of mouse body weight) b.i.d. for 2 weeks. For oral gavage, 14-week-old C57BL/6J DIO male mice were fed by oral gavage using a feeding needle attached to a sterile 1 mL syringe filled with 200 μL of methyl cellulose (MC, vehicle control) or 200 μL of NCT (200 mg/kg) dissolved in methyl cellulose twice a day for 2 weeks. All mice were maintained in a 12 h light/dark cycle throughout the experiment. For analysis all mice were sacrificed after 2 weeks of treatment.

Sample collection. Mouse samples were collected as described previously. Briefly, on the final day of treatment mice received dextrose (3 g/kg of body weight) by IP injection to stimulate insulin secretion, which inhibits FFA release from adipocytes, leaving liver-derived FFA as the major source of circulating FFA. One hour later, blood samples were collected via retro-orbital bleeding and mice were euthanized using pentobarbital. Mice were dissected aseptically and liver, epididymal fat, and body weights were measured and pictures taken. Dissected liver samples were washed immediately in sterile cold PBS and cut into small pieces. Half of the liver samples were snap frozen using liquid nitrogen and stored at −80° C. for RNA, protein isolation, and liver lysate preparations. The other half were fixed in 4% of cold paraformaldehyde (PFA, Santa Cruz Biotechnology, USA) and processed for histomorphometry and immunofluorescence.

Oil Red O staining (in vivo) and analysis. Oil red O staining was performed as described previously. Slides containing frozen liver tissue sections from mice were air dried for 10-20 min followed by rehydration in distilled water. Sections were immersed in absolute propylene glycol (Cat #151957, MP Biomedicals, LLC, USA) for 2 min followed by 0.5% in Oil red O solution (Cat #K043, Poly Scientific R&D, USA) for 2 h. Slides were then differentiated in 85% propylene glycol solution, washed with dH₂O for 2 h, and mounted using glycerin jelly mounting medium. All slides were scanned at a magnification of ×20 using the Aperio Scanscope FL system (Aperio Technologies Inc., Vista, CA, USA). The liver area stained with oil red O was measured using image J software as described, with some modifications as follow, Oil red O-stained liver images were opened in Image J software. Using the Analyze>Set Scale command, the scale bar of the images was set to 200 μm. RGB images were then converted into grayscale images using the Image>Type>RGB Stack command and were split into red, blue and green channels. Using the Image>Adjust>Threshold command, the threshold was manually set to highlight the Oil red O-stained lipid droplets in the green channel. The same threshold was used for all the images in all treatment groups and the % oil red O-stained area was obtained using the Analyze->Measure tool command. Fold change was calculated by normalizing the values to images from mice fed normal chow.

Triglyceride analysis. The TG level in mouse liver, serum, and stool was measured according to manufacturer's instructions using the Triglyceride calorimetric Assay Kit (Cat #10010303, Cayman Chemicals, USA). Liver and stool TG was normalized with liver and stool weight, respectively. Fold change was calculated by normalizing to values from mice fed NC.

Free fatty acid quantification. Blood samples were collected from dextrose injected mice and centrifuged to collect serum samples. The serum FFA level was measured according to manufacturer's instructions using a Free Fatty Acid Quantification Colorimetric/Fluorometric Kit (Cat #K612, BioVision, USA). Fold change was calculated by normalizing to values from mice fed NC.

Immunofluorescence. Frozen liver sections were permeabilized using 0.3% Triton-X and incubated in antigen retrieval solution (Antigen retrieval citrate, Biogenex) at sub-boiling temperature for 10 min. Subsequently, sections were incubated with blocking buffer containing 5% normal donkey serum (Jackson Immuno Research) followed by incubation overnight at 4° C. with mouse anti-HNF4α monoclonal antibody (1:800, Cat #PP-H1415-00, R&D Systems), rabbit polyclonal anti-VDAC antibody (1:400, cat #PA1-954A, Invitrogen) and cleaved caspase3 antibody (1:500, cat #9664, Cell Signaling). Sections were washed and incubated for 1 h at room temperature with anti-mouse secondary antibody coupled with Alexa fluor 488 (1:400, Invitrogen) and anti-rabbit secondary antibody coupled with rhodamine red. Nuclei were visualized by counterstaining with DAPI (40,6-diamidino-2-phenylindole, Sigma Aldrich). Slides were mounted using fluorescence mounting medium and images were obtained at ×40 magnification using an Olympus IX71 fluorescence microscope. Fluorescence intensity of HNF4α-stained nuclei and VDAC stained mitochondria was calculated using MetaMorph TL software (version 7.6.5.0, Olympus). Fold change was calculated by normalizing to values from mice fed NC.

Liver profile analysis. Mice were anesthetized and 100 μL of whole blood was collected via retro-orbital bleeding in lithium heparin blood collection tubes and transferred to single use VetScan mammalian liver profile reagent rotors. The levels of multiple analytes, including alkaline phosphatase (ALP), alanine aminotransferase (ALT), gamma glutamyl transferase (GGT), bile acids (BA), total bilirubin (TBIL), albumin (ALB), blood urea nitrogen (BUN), total cholesterol (CHOL) were quantified using a VetScan VS2 Chemistry Analyzer (Abaxis North America, USA).

Blood count analysis. Mice were anesthetized and 20 μL of whole blood was collected via retro-orbital bleeding in lithium heparin blood collection tubes and 20 different hematologic parameters were measured using a Hemavet 950 FS blood count analyzer (Drew Scientific Group).

Western blotting. Mouse liver extracts were prepared by incubation in RIPA buffer (Invitrogen) containing protease inhibitors (Calbiochem, SanDiego, CA). Protein was quantified by BCA assay (Thermo Scientific). Protein (40 or 80 mg for cytochrome C) was separated on 12 or 16% Tri-Glycine gels (Invitrogen) and transferred to Immobilon P membrane (0.2 μm pore size, Millipore). After 1 h in phosphate-buffered saline Tween (PBST) with 3% milk, membranes were incubated with antibodies to PPARGC1A(PGC1a) (Cat #NBP1-04676, Novus, 1:1000), SDHA XP (Cat #11998, Cell Signaling 1:1000), or Cytochrome C (Cat #4280, Cell Signaling 1:500), followed by secondary antibody conjugated to horse-radish peroxidase (1:5000, Jackson Immune or Cell Signaling). Signal was revealed by ECL (Thermo) and imaged with a ChemiDoc MP imager (Bio-Rad). After detection, membrane was incubated with Ponceau S solution (Sigma) for 1 h for normalization to loaded protein.

Primary human hepatocytes. Experiments with primary human hepatocytes were performed by CN-Bio (Cambridge, UK). Briefly, primary human hepatocytes (PHHs), human Kupffer cells (HKs) and human stellate cells (HSCs) were seeded onto CN-Bio's PhysioMimix LC12 MPS culture plates at 6×10⁵ cells for PHHs and 6×10⁴ cells for HKs and HSCs in 1.6 ml of CN-Bio's HEP-lean media with 5% FCS. Throughout the experiment the cells were maintained at a flow rate of 1 μl/s. After 24 hours (Day 1) of seeding, the media was changed to HEP-lean media and the cells were incubated until day 4 to allow the cells to form microtissues. At day 4 post seeding, media was changed to HEP-fat media and treated with DMSO or NCT (5, 15, and 40 μM). Media was replaced on days 6 and 8. Cells were harvested on day 10 for RNA extraction and culture media was collected for ELISA analysis.

RT-PCR. Total RNA was isolated from liver tissues and primary human hepatocytes using Trizol (Invitrogen). cDNA was amplified using 3 μg of total RNA using qScript cDNA SuperMix (Quanta BioSciences, Beverly, MA, USA). Quantitative real time PCR (RT-PCR) analysis was performed using SYBR® Select Master Mix (Applied Biosystems) and an ABI 7900HT thermal cycler (Applied Biosystems, Thermo Fisher Scientific). Ct values were normalized to 18s rRNA and are expressed as fold change over samples from mice fed NC or cultured human hepatocytes without NCT.

Fatty acid oxidation assay. FAO in liver lysate was measured according to manufacturer's instructions using a calorimetric assay kit (Cat #E-141, Biomedical Research Service Center, State University of New York, Buffalo, NY).

Nicotinamide adenine dinucleotide assay. Total NAD level in liver lysate was analyzed according to manufacturer's instructions using a calorimetric NAD+/NADH assay kit (Cat #MET-5014, Cell Biolabs, Inc. USA).

Citrate synthase activity. Citrate synthase activity in liver homogenate was measured according to manufacturer's instructions using a calorimetry based MitoCheck Citrate Synthase Activity Assay Kit (Cat #701040, Cayman Chemicals, USA).

Bicinchoninic acid (BCA) assay. A BCA protein assay was performed according to manufacturer's instructions using a kit from Thermo Scientific (Cat #23225). BCA assay was used for protein quantification for the FAO assay and Western blotting. Absorbance at 550 nm was determined using a plate reader.

### Example 2 Nitric Oxide Assay

T6PNE cells were maintained in RPMI (5.5 mM glucose, Corning) supplemented with 10% fetal bovine serum (FBS, Sigma-Aldrich) and 1% penicillin-streptomycin (pen-strep, Gibco) in 5% CO₂ at 37° C. Cells were treated with 0.12 μM palmitate plus 0 or 15 μM NCT for 3 days in 10 cm plates and harvested with 500 μL PBS. For tissue specimens, snap frozen mouse liver was weighed and homogenized with PBS. NO was measured with the QuantiChrom Nitric Oxide Assay kit (D2NO-100, BioAssay Systems). Homogenized samples (150 μL) were processed for deproteination with 8 μL ZnSO₄ and 8 μL NaOH. For normalization, an aliquot of samples from T6PNE cells was taken for BCA assay before deproteination. Samples and standard from kit were incubated with reagents for 20 min at 60° C. and measured OD 540 nM.

### Example 3 Palmitate-BSA Complex

150 mM Palmitate (Sigma-Aldrich) was prepared in 50% ethanol and precomplexed with 15% fatty acid-free BSA (Research Organics, Cleveland, OH, USA) in a 37° C. water shaker. BSA-precomplexed palmitate was used as a 12 mM stock solution for all assays with a final concentration of 0.12 mM palmitate in cell culture medium.

### Example 4 Mitochondrial DNA Analysis

Quantification of mtDNA was performed. Snap frozen mouse liver was homogenized and total cellular DNA was extracted with a QIAamp DNA kit (Qiagen) followed by qPCR with the primers disclosed in Veeriah, V., Lee, S H. & Levine, F. Long-term oral administration of an HNF4α agonist prevents weight gain and hepatic steatosis by promoting increased mitochondrial mass and function. Cell Death Dis 13, 89 (2022) (doi.org/10.1038/s41419-022-04521-5) which is incorporated herein by reference in its entirety.

### Example 5 Statistical Analysis

Data were presented as mean±SEM of three or more samples as indicated. Statistical significance was assessed using Student's t-test or ANOVA.

### Example 6 Compound of NCT was Evaluated for HFD Induced Weight Gain and Hepatic Steatosis

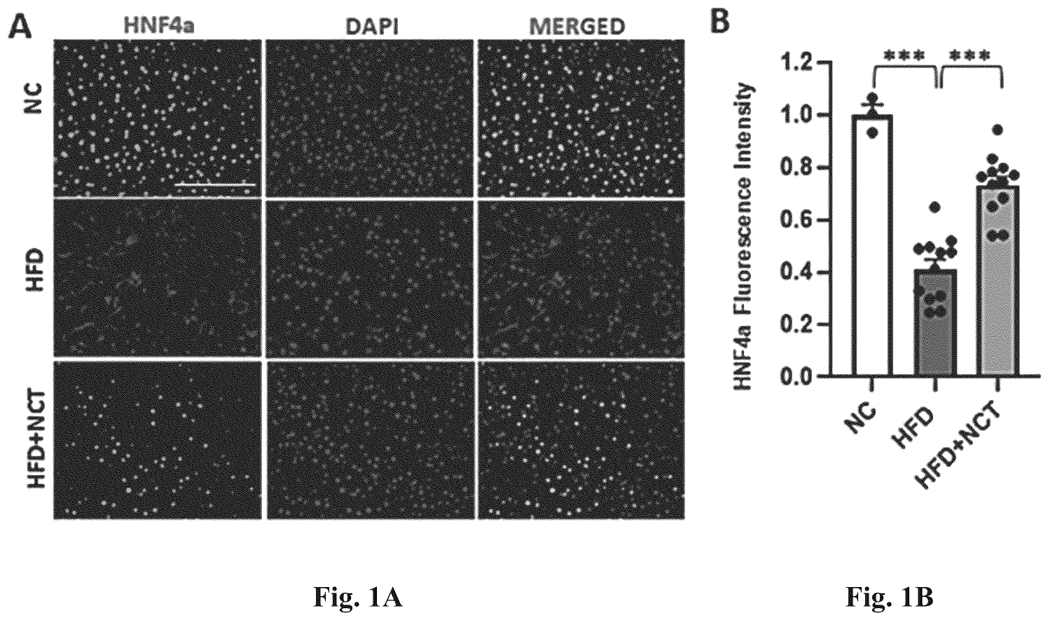

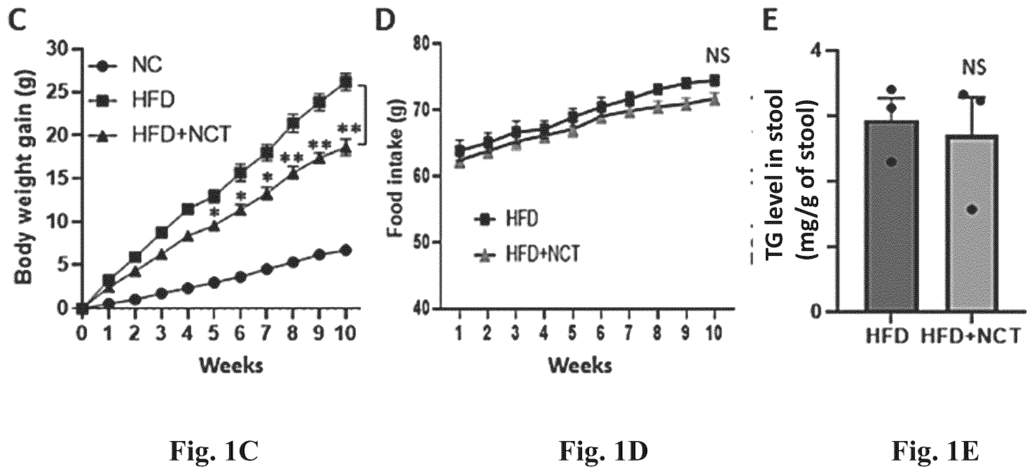

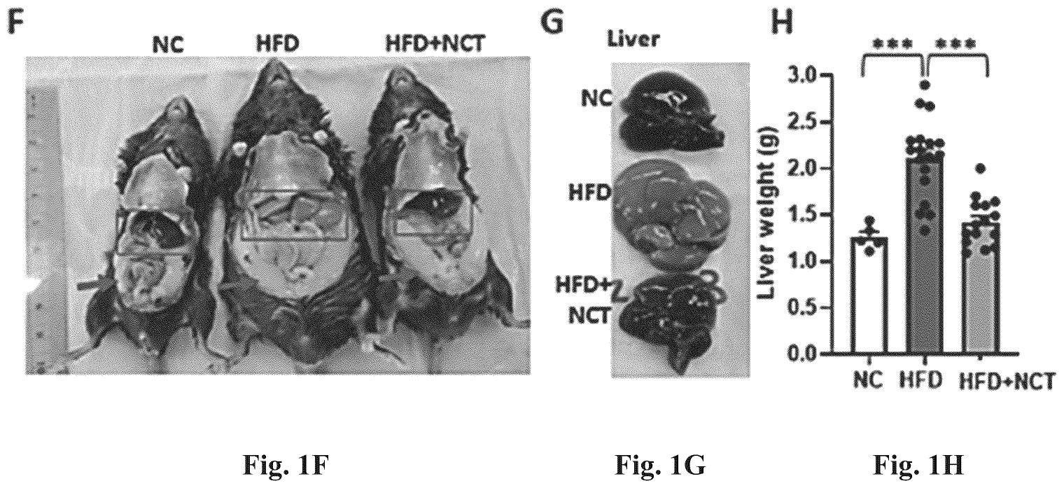

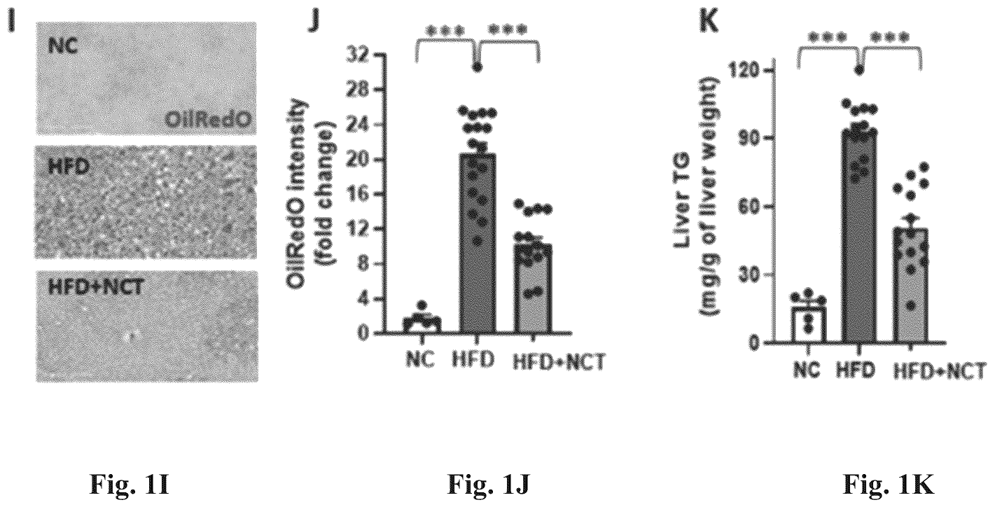

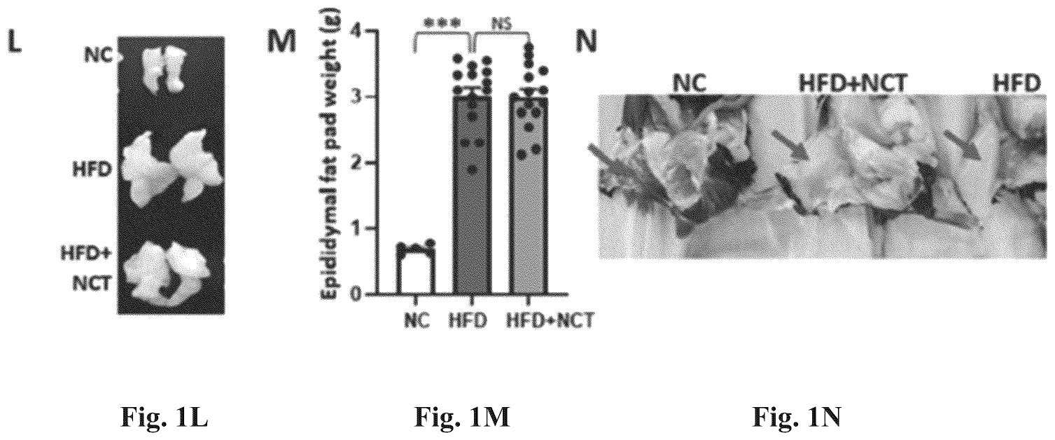

C57BL/6 mice were fed normal chow (NC), a high fat diet (HFD), or a high fat diet containing 4000 ppm NCT (HFD+NCT) for 10 weeks. Liver sections from mice fed NC, HFD, or HFD+NCT were immune stained for HNF4α (green nuclear staining) and DAPI (blue nuclear staining) as illustrated in FIG. 1A. Also as illustrated in FIG. 1B, quantification of HNF4α fluorescence intensity from images stained as in FIG. 1A, after non-specific cytoplasmic staining was subtracted from the HNF4α nuclear staining. The fold change was calculated vs. NC (NC, N=3, HFD and HFD+NCT, N=12). HFD led to decreased expression of HNF4α, but this was reversed by NCT as illustrated in FIG. 1A and FIG. 1B. Thus, oral administration of NCT at approximately the same dose as IP administration was effective at stimulating HNF4α activity.

Body weight was measured each week for 10 weeks as illustrated in FIG. 1C. Body weight gain was calculated by subtracting the baseline body weight at the start of the study (NC N=5, HFD and HFD+NCT, N=15). It became evident at week 5 that mice fed HFD+NCT weighed less than mice fed HFD alone as shown in FIG. 1C. By the end of the experiment at 10 weeks, the mice fed HFD+NCT weighed ˜10 g less than the mice fed HFD, a 35-40% difference in body weight. However, as illustrated in FIG. 1D, HFD and HFD+NCT chow consumption per cage was measured every week for 10 weeks, no difference was found between the two groups (five cages for each condition, three mice in each cage). Thus, indicating the NCT was not aversive and did not cause the mice to become ill and consume less chow. Further, as illustrated in FIG. 1E, levels of triglyceride (TG) in the stool were equal in the HFD and HFD+NCT groups, ruling out the possibility of fat malabsorption as being responsible for the difference in weight. TG was normalized to the stool dry weight (N=3).

The HFD+NCT mice were markedly less obese as illustrated in FIG. 1F, with substantially less subcutaneous fat as shown in FIG. 1N, but no change in epididymal fat pad weight at the end of the study as shown in FIG. 1L and quantified in FIG. 1M. As shown in FIGS. 1F and G, mice treated with HFD+NCT demonstrated reductions in visceral adiposity and liver size and an increase in liver redness. In FIG. 1F, boxes indicate liver and the arrows indicate epididymal fat pad. In FIG. 1M, epididymal fat pad weight was quantified by normalizing with total mouse body weight (NC, N=5, HFD and HFD+NCT, N=15). Dots indicate individual mice. No difference in epididymal fat pad weight was observed between HFD and HFD-NCT groups. As illustrated in FIG. 1N, mice treated with HFD-NCT demonstrated a reduction in subcutaneous fat (indicated by arrows).

As illustrated in FIG. 1G, HFD+NCT mice had redder livers, and lower liver weight as shown in FIG. 1H. Consistent with the increased redness and decreased weight, the HFD+NCT livers exhibited decreased Oil Red O staining as shown in FIG. 1I, and as quantified in FIG. 1J. Similarly, HFD+NCT treatment decreased levels of TG as shown in FIG. 1K. As shown in FIG. 1J, quantification of Oil red O staining HFD and HFD+NCT values were measured using image in FIG. 1J, with consistent threshold settings and normalized to NC values to calculate fold change (NC, N=5, HFD and HFD+NCT, N=15). As shown in FIG. 1K, hepatic TG level was normalized to the liver weight (NC, N=5, HFD and HFD+NCT, N=15). The above results suggest that NCT reduces HFD induced weight gain and hepatic steatosis.

### Example 7 Compound of NCT was Evaluated for Increased Hepatic Mitochondrial Mass

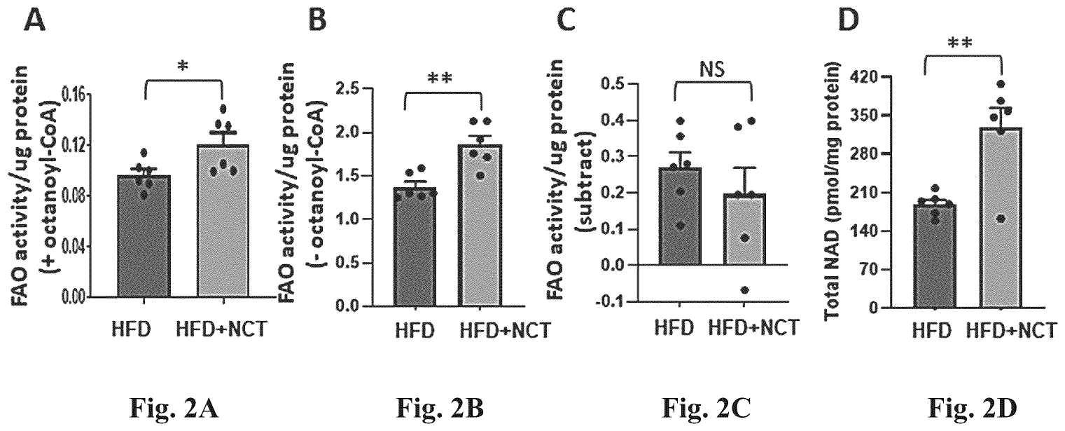

As illustrated in FIG. 2A, NCT induced an increase in FAO activity in the presence of octanoyl CoA. However, there was also an increase in the baseline activity in the absence of octanoyl CoA as shown in FIG. 2B, so that the overall activity in the assay was unchanged as shown in FIG. 2C. Because this assay is sensitive to the cellular mitochondrial mass, this suggested that the effect of NCT might be on mitochondrial mass rather than specifically on fatty acid oxidation, i.e., NCT might be acting to stimulate increased mitochondrial mass with a consequent increase in total fatty acid oxidation without stimulating an increase in the level of fatty acid oxidation per unit of mitochondrial mass. Independent measurement of total cellular NAD, the majority of which is in the mitochondria, revealed a substantial increase of NAD in the livers of mice treated with NCT as illustrated in FIG. 2D. Thus, the effect of NCT on FAO appeared to be a secondary rather than primary effect.

The examples and embodiments described herein are for illustrative purposes only and various modifications or changes suggested to persons skilled in the art are to be included within the spirit and purview of this application and scope of the appended claims.

1. A method for reducing body weight of a subject, comprising administering N-trans caffeoyltyramine, or a pharmaceutically acceptable salt thereof, to the subject in an amount of 30 mg per kg per day to about 400 mg per kg per day for a period of time.

2. A method for reducing body weight and treating nonalcoholic fatty liver disease (NAFLD), treating nonalcoholic steatohepatitis (NASH), or treating diabetes in a subject in need thereof, comprising administering N-trans caffeoyltyramine, or a pharmaceutically acceptable salt thereof, to the subject in an amount of 30 mg per kg per day to about 400 mg per kg per day for a period of time.

3. (canceled)

4. (canceled)

5. The method of claim 1, wherein administering N-trans caffeoyltyramine, or a pharmaceutically acceptable salt thereof, to the subject in an amount of 30 mg per kg per day to about 400 mg per kg per day for a period of time increases fatty acid oxidation (FAO) activity in the subject.

6. (canceled)

7. The method of claim 1, wherein administering N-trans caffeoyltyramine, or a pharmaceutically acceptable salt thereof, to the subject in an amount of 30 mg per kg per day to about 400 mg per kg per day for a period of time reduces mitochondrial stress in the subject.

8. The method of claim 1, wherein administering N-trans caffeoyltyramine, or a pharmaceutically acceptable salt thereof, to the subject in an amount of 30 mg per kg per day to about 400 mg per kg per day for a period of time treats inflammation in the subject.

9. The method of claim 8, wherein the level of IL-6, TNFα, or nitric oxide in the subject is decreased.

10. (canceled)

11. (canceled)

12. A method for maintaining weight of a subject or reducing diet induced weight gain of a subject and treating nonalcoholic steatohepatitis (NASH) in a subject in need thereof, comprising administering N-trans caffeoyltyramine, or a pharmaceutically acceptable salt thereof, to the subject in an amount of 30 mg per kg per day to about 400 mg per kg per day for a period of time.

13. (canceled)

14. (canceled)

15. (canceled)

16. (canceled)

17. (canceled)

18. (canceled)

19. The method of claim 1, wherein the N-trans caffeoyltyramine, or a pharmaceutically acceptable salt thereof is administered orally.

20. The method of claim 19, wherein the N-trans caffeoyltyramine, or a pharmaceutically acceptable salt thereof, is administered once a day (q.d.), twice a day (b.i.d.), three times a day (t.i.d.), or four times a day (q.i.d.).

21. The method of claim 20, wherein the period of time is at least 2, 3, 4, 5, 6, 7, 8, 9, 10, 11, or 12 weeks, or a period of time within a range defined by any of the preceding values, or wherein the period of time is at least 1, 2, 3, 4, 5, 6, 7, 8, 9, 10, 11, or 12 months, or a period of time within a range defined by any of the preceding values.

22. (canceled)

23. The method of claim 21, wherein the subject is having a high fat diet (HFD).

24. The method of claim 23, wherein the subject has obesity.

25. The method of claim 25, wherein the subject has a BMI of at least about 25, about 26, about 27, about 28, about 29, about 30, or about 32.

26. The method of claim 25, wherein the body weight of the subject is reduced by at least about 10%, 20%, 30%, 40%, or 50%, or reduced an amount by weight % within a range defined by any of the preceding values, as compared to a reference subject having a similar body weight and the same diet but is not administered N-trans caffeoyltyramine, or a pharmaceutically acceptable salt thereof.

27. The method of 26, wherein mitochondrial mass and/or function is increased in the subject.

28. The method of claim 27, wherein the mitochondrial mass/or function is measured by the expression level of cytochrome C or succinate dehydrogenase (SDHA).

29. The method of claim 28, wherein the mitochondrial mass and/or function is increased in the subject's liver.

30. The method of claim 29, wherein the mitochondrial mass and/or function is increased in primary hepatocytes of the subject.

31. (canceled)

32. The method of claim 30, further comprising measuring the expression level of VDAC1, citrate synthase, sirtuin, or PPARGC1A (PGC1α).

33. (canceled)

34. (canceled)

35. The method of claim 1, wherein the subject is a human.

36. (canceled)
